(19) United States
(12) Patent Application Publication
(10) Pub. No.: US 2011/0320392 A1
(43) Pub. Date: Dec. 29, 2011

(54) METHODS FOR PREDICTING CANCER RESPONSE TO EGFR INHIBITORS

(76) Inventors: Esther P. Black, Lexington, KY (US); Justin M. Balko, Nashville, TN (US)

(21) Appl. No.: 13/254,663

(22) PCT Filed: Mar. 2, 2010
(86) PCT No.: PCT/US10/25918

§ 371 Date: Sep. 2, 2011

Related U.S. Application Data
(60) Provisional application No. 61/156,825, filed on Mar. 2, 2009.
(60) Provisional application No. 61/250,183, filed on Oct. 9, 2009.

(51) Int. Cl. G06F 15/18

(52) U.S. Cl. 706/21

(57) ABSTRACT

The presently-disclosed subject matter relates to biomarker profiling of samples obtained from carcinoma subjects who are candidates for treatment with a therapeutic EGFR inhibitor. More specifically, the presently-disclosed subject matter provides methods of biomarker profiling which allow one skilled in the art to predict whether a patient is likely to respond well to treatment with an EGFR inhibitor.

### RELATED APPLICATIONS

This application claims priority from U.S. Provisional Application Ser. Nos. 61/250,183 filed Oct. 9, 2009, and 61/156,825 filed on Mar. 2, 2009, the entire disclosures of which are incorporated herein by this reference.

### GOVERNMENT INTEREST

Subject matter described herein was made with U.S. Government support under Grant Number 2P20 RR-16481 awarded by the National Center for Research Resources of the National Institutes of Health. The government has certain rights in the described subject matter.

### TECHNICAL FIELD

The presently-disclosed subject matter relates to biomarker profiling of samples obtained from carcinoma subjects who are candidates for treatment with a therapeutic EGFR inhibitor. More specifically, the presently-disclosed subject matter provides methods of biomarker profiling which allow one skilled in the art to predict whether a patient is likely to respond well to treatment with an EGFR inhibitor.

### INTRODUCTION

A significant challenge in oncology practice is determining the best therapeutic regimen to offer individual patients. The epidermal growth factor receptor (EGFR) is an appealing target for novel therapies for cancer because it plays a major role in transmitting stimuli that lead to proliferation, growth and survival of various cancer types. EGFR inhibitors have been approved for treatment of various epithelial cancers, or carcinomas; however, success has been limited due to the significant population of non-responders.

Several groups have investigated potential biomarkers for predicting carcinoma patient's response to EGFR inhibitors (see for example, US 20050164218 to Agus et al.; US 20060252056 to Tsuruo et al; US 20070212738 to Haley et al.; US 20080113874 to Bunn et al.; US 20080286771 to Hudson et al.; US 20080318230 to Agus et al.). However, no diagnostic or prognostic tests have yet emerged that can effectively guide medical practitioners in the treatment of their patients with EGFR inhibitors.

Mutations in the KRAS gene have been correlated with non-response of carcinomas to EGFR inhibitors; however, a large percentage of KRAS wild-type patients also do not realize benefit from EGFR inhibitors (see for example, Karapetis et al. (2008); Khambata-Ford et al. (2007). Therefore, additional methods of patient stratification are required to improve the tailoring of EGFR-targeted therapy in these patients. Previously, a 180-gene panel was used to predict sensitivity of cultured non-small cell lung cancer cells to erlotinib in vitro, an EGFR inhibitor (Balko et al. (2006)).

There remains a need in the art for profiling methods of predicting a therapeutic response of a subject with a carcinoma to EGFR inhibitors.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes a method, device, and kit for predicting clinical outcome for a subject with a carcinoma.

In some embodiments, the carcinoma is colorectal cancer, pancreatic cancer, or head and neck cancer. In some embodiments, the carcinoma is colorectal cancer. In some embodiments, the carcinoma is not a non-small cell lung carcinoma.

In some embodiments, the clinical outcome is EGFR inhibitor sensitivity of the carcinoma. In some embodiments, the clinical outcome is sensitivity to an EGFR inhibitor that is an antibody. In some embodiments, the clinical outcome is sensitivity to an EGFR inhibitor that is a small molecule. In some embodiments, the clinical outcome is survival.

In some embodiments, the method includes: determining an RNA expression profile in the carcinoma of the subject; and applying an algorithm for predicting a clinical outcome indicator from an RNA expression profile of a carcinoma to the RNA expression profile of the subject to predict a clinical outcome indicator of the subject, wherein the RNA expression profile comprises at least two predictive markers.

In some embodiments, the method also includes applying a second algorithm for predicting a second clinical outcome indicator from the RNA expression profile of a carcinoma to the RNA expression profile of the subject to predict a second clinical outcome indicator of the subject. In some embodiments, the first clinical outcome indicator is EGFR inhibitor sensitivity or survival and wherein the second clinical indicator is EGFR inhibitor sensitivity or survival and wherein the first and second clinical outcome indicators are not the same clinical outcome indicator.

In some embodiments, the RNA expression profile comprises two to about 170 predictive markers. In some embodiments, the RNA expression profile comprises about 10 to about 100 Predictive Markers. In some embodiments, the RNA expression profile comprises about 2 to about 50 Predictive Markers.

In some embodiments, the predictive markers are messenger RNA (mRNA) molecules. In some embodiments, the predictive markers are microRNA (miR) molecules. In some embodiments, the predictive markers comprise two or more predictive markers from Table 1. In some embodiments, the predictive markers comprise two or more predictive markers from Table 5. In some embodiments, the predictive markers comprise two or more predictive markers from Table 1 and Table 5.

In some embodiments, the algorithm, when applied to a plurality of subjects, yields predicted clinical outcome indicators that correlate to a statistically significant extent with the actual clinical outcomes.

In some embodiments, determining the RNA expression profile in a carcinoma of the subject comprises determining an amount in a biological sample from the subject of at least two predictive markers.

In some embodiments, the method further includes using probes for the predictive markers. In some embodiments, the probes are provided in a device.

In some embodiments, the device for predicting a clinical outcome indicator for a carcinoma includes probes for predictive markers for determining an RNA expression profile in a carcinoma of a subject.

In some embodiments, the device further includes an algorithm for predicting a clinical outcome indicator from an RNA expression profile of a carcinoma by applying the algorithm to the RNA expression profile in a carcinoma of the subject, wherein the RNA expression profile comprises at least two predictive markers.

In some embodiments, the device further includes a second algorithm for predicting a second clinical outcome indicator from the RNA expression profile of a carcinoma by applying the second algorithm to the RNA expression profile of the subject to predict a second clinical outcome indicator of the subject. In some embodiments, the first clinical outcome indicator is EGFR inhibitor sensitivity or survival and wherein the second clinical indicator is EGFR inhibitor sensitivity or survival and wherein the first and second clinical outcome indicators are not the same clinical outcome indicator.

In some embodiments, the device includes two to about 170 probes for predictive markers. In some embodiments, the device includes about 10 to about 100 probes for predictive markers. In some embodiments, the device includes 2 to about 50 probes for predictive markers.

In some embodiments, the probes are for predictive markers that are RNA molecules. In some embodiments, the RNA molecules are selected from messenger RNA (mRNA) molecules and microRNA (miR) molecules. In some embodiments, the RNA molecules are messenger RNA (mRNA) molecules. In some embodiments, the RNA molecules are microRNA (miR) molecules. In some embodiments, the probes are for predictive markers comprising two or more predictive markers from Table 1. In some embodiments, the probes are for predictive markers comprising two or more predictive markers from Table 5. In some embodiments, the probes are for predictive markers comprising two or more predictive markers from Table 1 and Table 5.

In some embodiments, the algorithm, when applied to a plurality of subjects, yields predicted clinical outcome indicators that correlate to a statistically significant extent with the actual clinical outcomes.

In some embodiments, the device further includes instructions for determining the RNA expression profile in a carcinoma of the subject.

In some embodiments, the device is a qRT-PCR based device.

In some embodiments, the device further includes probes for KRAS status.

In some embodiments, the kit for predicting a clinical outcome indicator for a carcinoma includes an algorithm for predicting a clinical outcome indicator from an RNA expression profile of a carcinoma by applying the algorithm to an RNA expression profile in a carcinoma of a subject, wherein the RNA expression profile comprises at least two predictive markers.

In some embodiments, the kit also includes probes for predictive markers for determining the RNA expression profile in the carcinoma of the subject.

In some embodiments, the kit further includes a second algorithm for predicting a second clinical outcome indicator from the RNA expression profile of a carcinoma by applying the second algorithm to the RNA expression profile of the subject to predict a second clinical outcome indicator of the subject. In some embodiments, the first clinical outcome indicator is EGFR inhibitor sensitivity or survival and wherein the second clinical indicator is EGFR inhibitor sensitivity or survival and wherein the first and second clinical outcome indicators are not the same clinical outcome indicator.

In some embodiments, the kit also includes instructions for determining the RNA expression profile in a carcinoma of the subject.

In some embodiments, the kit includes probes for predictive markers for determining the RNA expression profile in the carcinoma of the subject

### BRIEF DESCRIPTION OF THE DRAWINGS

FIG. 1 shows the EGFR inhibitor response classification in subjects with colorectal cancer based upon gene expression profile (top panel, KRAS-wildtype patients; middle panel, all patients; bottom panel, KRAS-mutant patients), (NA, no survival data; PD, progressive disease; SD, stable disease; CR/PR, complete response/partial response).

FIG. 2 shows stratification of KRAS-wildtype patients by 180-gene Predictive Marker profile.

FIG. 3 show stratification of all patients by 180-gene Predictive Marker profile

FIG. 4 shows stratification of KRAS-mutant patients by 180-gene Predictive Marker profile.

FIG. 5 shows heatmap of signal intensities for the 26-gene Predictive Marker profile for all patients with KRAS mutant status identified by arrows.

FIG. 6 shows a plot of KRAS-WT samples by predicted sensitivity (bottom panel) and Kaplan-Meier survival plot of PFS between the ‘sensitive’ and ‘resistant’ groups

FIG. 7 shows stratification of KRAS wildtype patients by 15-gene Predictive Marker profile

FIG. 8 shows stratification of all patients by 15-gene Predictive Marker Profile

FIG. 9 shows stratification of KRAS-mutant patients by 15-gene Predictive Marker Profile.

FIG. 10 is a schematic representation of the analysis of data to identify and validate miR predictive markers.

FIG. 11 shows hierarchical clustering of expression data from 13 differentially-expressed miRs from cancer cell lines and tumors.

FIG. 12 shows a DLDA prediction of sensitivity to erlotinib in cancer cells/tumors.

FIG. 13 shows the biological significance (target, Zeb1) of one of the 13 differentially-expressed miRNA in lung and pancreatic cell lines (miR-200c).

### DESCRIPTION OF EXEMPLARY EMBODIMENTS

The presently-disclosed subject matter includes methods, devices, and kits for predicting a clinical outcome in a subject having a carcinoma. The presently-disclosed subject matter makes use of profiles of predictive markers to obtain clinically-relevant information, which can be used, for example, in making decisions concerning treatment of a subject.

By analyzing key predictive markers in a biological sample from a subject and comparing it to the profiles of biological samples from subjects with known cancer phenotypes, useful information can be obtained. In some embodiments, the information can be used to identify subjects that will likely be responsive to small molecule EGFR inhibitors, antibody EGFR inhibitors, or combinations thereof.

While clinical utility of gene expression profiling has been suggested in the past or performed under certain conditions, the value of certain predictive marker combinations across carcinoma types, as described herein, was not expected. Moreover, it could not have been expected that biomarker profiling, as described herein, would be useful for such a substantial portion of subjects with a carcinoma.

In some embodiments of the presently-disclosed subject matter, a method is provided for predicting clinical outcome for a subject with a carcinoma, which includes: determining a biomarker profile in the carcinoma of the subject, and applying an algorithm for predicting a clinical outcome indicator from a biomarker profile to the biomarker profile of the subject to product a clinical outcome indicator of the subject, where the biomarker profile comprises at least two predictive markers.

As used herein the term “profile” refers to measurements made from a plurality of Predictive Markers from a biological sample containing cancer cells or cancer cell products.

As used herein, the term “clinical outcome indicator” means an indicator of clinical outcome such as survival or sensitivity to an EGFR inhibitor as described herein. Depending upon the context, Clinical outcome indicator can refer to the variable itself or to the value established or predicted for a given subject.

As used herein, the term “predictive markers” refers to biomarkers described herein that correlate or are statistically associated with a clinical outcome of a cancer. As such, predictive markers can be used in a biomarker profile. A predictive marker can be, for example, a gene, an RNA gene product, a protein gene product, a microRNA, or a probe (e.g. antibody or polynucleic acid) for a predictive marker. In some embodiments, the predictive markers can be RNA molecules, and the profile can be an RNA expression profile. Certain messenger RNA (mRNA) molecules and/or microRNA (miR or miRNA) molecules can serve as predictive markers in an RNA expression profile.

In some embodiments, the predictive markers can comprise two or more mRNA molecules as set forth in Table 1. In some embodiments, the predictive markers can comprise two or more miR molecules as set forth in Table 5. In some embodiments, the predictive markers can comprise a combination of mRNA molecules and miR molecules.

The expression profiles of Predictive Markers are useful for determining efficacy of EGFR inhibitors for a given subject. In some embodiments, a method is provided for identifying subjects that will likely be responsive to small molecule EGFR inhibitors, antibody EGFR inhibitors, or combinations thereof. In some embodiments, a method of predicting disease progression is provided. In some embodiments, the subject is administered treatment according to the prediction of efficacy of an EGFR inhibitor.

Examples of EGFR inhibitors that pertain to the presently-disclosed subject matter include a therapeutic antibody, for example, anti-human EGFR (anti-HER1) antibody panitumumab (VECTIBIX®, AMGEN, Thousand Oaks, Calif.), or cetuximab (ERBITUX®, ImClone Systems/Bristol-Myers Squibb).

An EGFR inhibitor can be a small molecule inhibitor of a target within the EGFR pathway. Examples of such small molecule inhibitors are gefitinib (Iressa®, AstraZeneca, Wilmington, Del.) erlotinib (Tarceva®, OSI Pharmaceuticals Inc, Melville, N.Y.), PKI-166; EGFR-specific and irreversible inhibitors, such as EKI-569; a PAN-HER (human EGF receptor family) reversible inhibitor, such as GW2016 (targets both EGFR and Her2/neu); and a PAN-HER irreversible inhibitor, such as CI-1033 (4-anilinoquinazoline).

An EGFR inhibitor can be a broad-spectrum tyrosine kinase inhibitor such as lapatinib or canertinib, which have activity on more members of the ErbB family of receptors, and ZD6474 and AEE788, and EGFR.

An EGFR inhibitor can be an RNAi construct directed against a target within the EGFR pathway.

The term “EGFR inhibitor” refers to an agent that results in blunting of an aspect of the EGFR pathway. For example, an EGFR can embrace an inhibitor of EGFR tyrosine kinase or down stream affects of EGFR phosphorylation. For another example, an EGFR inhibitor can have an ability to inhibit an EGFR function, immediate or downstream therefrom. For another example, inhibitors useful in the presently-disclosed subject matter include agents that directly target the EGFR itself. In other embodiments, the agent useful as an EGFR inhibitor interacts with other members of the EGFR signal transduction pathway.

EGFR inhibitors useful in the presently-disclosed subject matter include nanoparticle-encapsulated therapeutics which are tethered to EGFR-targeting antibodies or EGFR-ligands, such as transforming growth factor-alpha, epidermal growth factor, betacellulin, amphiregulin, epiregulin, or heparin binding epidermal growth factor-like ligand.

EGFR inhibitors useful in the presently-disclosed subject matter include agents which bind and/or target EGFR ligands, such as transforming growth factor-alpha, epidermal growth factor, betacellulin, amphiregulin, epiregulin, or heparin binding epidermal growth factor-like ligand. Example—neutralizing antibody for EGF or TGFA.

EGFR inhibitors useful in the presently-disclosed subject matter include agents which inhibit the enzymatic processing and activation of EGFR ligands, e.g. ADAM17 inhibitor.

One indicator of clinical outcome that is described herein is “sensitivity of a cancer to treatment with an epidermal growth factor receptor (EGFR) Inhibitor”. According to the presently-disclosed subject matter, carcinomas can be classified as responsive or non-responsive or can be expressed using some other quantitative or semi quantitative metric.

In accordance with the presently-disclosed subject matter, clinical outcome, such as survival of a subject in response to an EGFR inhibitor can be predicted. In some embodiment, clinical outcome can be prognosis of a subject in response to an EGFR inhibitor; survival of a subject in response to an EGFR inhibitor; overall survival; disease-free survival, e.g., the period that the subject remains free of disease after treatment; or progression-free survival, e.g. the period that the subject remains stable (without signs of progression) at a specified time after treatment. Another indicator of clinical outcome, as disclosed herein, is Response Evaluation Criteria in Solid Tumours (RECIST) response.

EGFR inhibitor sensitivity information can be obtained in any useful method known to the skilled artisan. For example, sensitivity can be accessed from historical data (e.g. from the subject's medical charts) or can be obtained by in vitro assay using cultured cancer cells from the subject.

For example, a subject's response to an EGFR inhibitor can be estimated by radiographic progression of the cancer after initiation of the inhibitor (e.g. using the RECIST criteria; e.g. Therasse et al.)

For example, the response of a cultured cancer cells (e.g. cell lines) to an EGFR inhibitor can be estimated by treating cells at varying concentrations of the inhibitor and constructing a dose-response curve. IC50 (the concentration required to inhibit 50% of assay signal), GI50 (the concentration required to inhibit 50% growth of signal), or other variations thereof (i.e. IC90, GI90, TGI, etc.) can be interpolated from the dose response curves. Assays used to construct a dose response curve include, but are not limited to Lactate dehydrogenase assays, sulfarhodamine B assays, MTT assays, MTS assays, trypan blue assays, fluorescence-assisted cell sorting, clonogenic survival assays, and in vivo xenograft assays. See, e.g. Cheng and Prusoff.

For example, a cell line response to an EGFR inhibitor can be estimated by treating cells at a fixed clinically achievable concentration and determining the extent of change relative to control in assay signal. Assays used to measure the extent of change include, but are not limited to Lactate dehydrogenase assays, sulfarhodamine B assays, MTT assays, MTS assays, trypan blue assays, fluorescence-assisted cell sorting, clonogenic survival assays, and in vivo xenograft assays.

The Predictive Markers of the presently-disclosed subject matter relate to clinical outcome of subjects with cancers of epithelial origin (“carcinoma”). The carcinoma can be a lung cancer, skin cancer, colorectal cancer, breast cancer, pancreatic cancer, prostate cancer, ovarian cancer, head and neck cancer, esophageal cancer, glioblastoma multiforme, hepatocellular cancer, gastric cancer, laryngeal cancer, cervical cancer, liver cancer, bladder cancer, stomach cancer, intestinal cancer, uterine cancer, cancer of the urinary tract, thyroid cancer, renal cancer, carcinoma, melanoma, and/or brain cancer.

In some embodiments, cancers useful in profiling of the present invention are EGFR-expressing cancers, such as non-small cell lung cancer (NSCLC), colorectal cancer, breast cancer, pancreatic cancer, prostate cancer, ovarian cancer, head and neck cancer (including head and neck squamous cell carcinoma, SCCHN), esophageal cancer, and glioblastoma multiforme. In some embodiment, the carcinoma is a carcinoma other than a lung carcinoma. In some embodiment, the carcinoma is a carcinoma other than a non small cell lung cancer.

Biological samples of the presently-disclosed subject matter can be any sample that contains carcinoma cells or carcinoma products. Such samples can be obtained by any method of the skilled artisan. Examples of useful biological samples include, but are not limited to, biopsies from sample cancer tissue or from blood, plasma, serum, urine, sweat, saliva, or other bodily fluids or excretions that might contain cancer-derived Predictive Markers. Biological samples can also be cells cultured from samples obtained from a subject.

Treatment of cancer often involves resection of the cancer to the extent possible without severely compromising the biological function of the subject. As a result, cancer tissue is typically available for analysis following initial treatment of the cancer, and this resected cancer is often available for use as a biological sample in accordance with the presently-disclosed subject matter.

Cancer tissue obtained through other means such as core-needle biopsy, fine needle aspiration, bronchial lavage, pleural effusion, transbronchial biopsy, or other types of biopsy can serve as a biological sample for use in accordance with the presently-disclosed subject matter.

Particularly in relatively-advanced cancers, circulating cancer cells are sometimes found in the blood of cancer patients and can be recovered from blood and used in a biological sample in accordance with the presently-disclosed subject matter.

Cellular constituents, including RNA, miR, and protein derived from cancer cells have been found in biological fluids of cancer patients, including blood and urine. Circulating polynucleic acids and proteins may be used as a biological sample in accordance with the presently-disclosed methods. For example, cellular constituents useful for gene expression profiling include circulating tumor-derived, exosomal materials.

The biological sample can be subjected to a variety of post-collection preparative and storage techniques (e.g., lysis, nucleic acid and/or protein extraction, fixation, storage, freezing, filtration, ultrafiltration, concentration, evaporation, centrifugation, etc.) prior to assessing the amount of the marker in the sample.

Predictive Markers are selected according to the presently-disclosed subject matter to be used in profiling, e.g., RNA expression profiling, gene expression profiling, etc. In some embodiments, the profile is made up from less than all Predictive Markers described herein.

It has been discovered that Predictive Markers, when selected by methods described herein here, can be sufficient to provide clinically useful information.

By way of example, when the clinical outcome indicator is EGFR inhibitor sensitivity, the Predictive Markers are selected such that the there is a positive correlation with statistical significance between predicted sensitivity and actual sensitivity of comparator subjects.

When the clinical outcome indicator is survival, the Predictive Markers are selected such that the there is a positive correlation with statistical significance between predicted survival and actual survival of comparator subjects.

When the clinical outcome indicator is EGFR inhibitor sensitivity and where the sensitivity is expressed as a binary (i.e. sensitive or resistant), the Predictive Markers are selected such that the survival of the predicted EGFR inhibitor sensitive subjects is statistically greater than the EGFR inhibitor resistant patients.

When the clinical outcome indicator is EGFR inhibitor sensitivity and where the sensitivity is expressed as a binary (i.e. sensitive or resistant), the Predictive Markers are selected such that the proportion of predicted EGFR inhibitor sensitive subjects achieving a clinical response or stable disease is statistically greater than the EGFR inhibitor resistant patients.

In some embodiments, a profile is made from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 67, 68, 69, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, or 170 of the predictive markers of Table 1.

In some embodiments, a profile is made from 2, 3, 4, 5, 6, 7, 8, 9, 10, 22, 12, or 13 of the predictive markers of Table 5.

In some embodiments, a profile is made from a combination of predictive markers as set forth in Tables 1 and 5.

It has been surprisingly discovered that a sequential, multi-step process for selecting Predictive Markers for a profile can result in a achieving a high level of predictive value. Taught herein are methods of selecting Predictive Markers based upon analyses with multiple clinical outcome indicators and/or upon biological knowledge (gene ontology hierarchy).

It is useful to reduce complexities in any diagnostic kits. Complexities increase cost and have greater risk for technical failure. Additionally, profiles containing few Predictive Markers reduce the likelihood of “over-fitting” data. Surprisingly, the present invention solves these problems and discloses how to select subsets of Predictive Markers that retain clinically useful predictive power.

The usefulness of the profiles can be further enhanced by the addition of one or more additional markers known in the art to be correlated with sensitivity or insensitivity. Non-limiting examples include KRAS mutations and/or expression level, EGFR mutations and/or expression level or BRAF mutations and/or expression level. The presence additional markers may be detected using the methods provided herein or may already be known in the subject.

Mutations in the KRAS gene have been correlated with insensitivity of carcinomas to EGFR inhibitors, however, a large percentage of K-RAS wild-type patients do not realize benefit from EGFR inhibitors. Accordingly, some embodiments of the present invention further include analyses of K-RAS and/or EGFR genotype.

While the present invention does not depend upon any specific bioinformatic or statistical methods, one skilled in the art will recognize effective approaches from the disclosure here. For example, the methods taught by the inventors in a previous publication set forth successful approaches (see Balko et al.)

The expression levels of Predictive Markers are detected to provide a profile according to the presently-disclosed subject matter. Any quantitative or semi-quantitative means of detection which allows for discriminating between unlike expression levels can be utilized. The following are non-limiting examples of techniques that allow detection of a Predictive Marker expression level.

As will be understood by those skilled in the art, the steps of a representative protocol for profiling using predictive markers will differ depending on the predictive markers that are being used. For example, isolation, purification, amplification, and detection of particular RNA molecules will differ as between mRNA and miR molecules, and the detection methods that are used. In this regard, various methods known to those skilled in the art are contemplated for use in accordance with the presently-disclosed subject matter.

In some embodiments, a representative protocol for profiling gene expression using fixed, paraffin-embedded tissues as the RNA source, including RNA isolation, purification, primer extension and amplification can be conducted according to various published journal articles, for example: Godfrey et al. Briefly, a representative process starts with cutting about 10 μm thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by Reverse Transcriptase PCR (RT-PCR). Finally, the data are analyzed to identify the best treatment option(s) available to the patient on the basis of the characteristic gene expression pattern identified in the tumor sample examined.

Methods of gene expression profiling include methods based on hybridization analysis of polynucleotides, methods based on sequencing of polynucleotides, and proteomics-based methods. The most commonly used methods known in the art for the quantification of RNA expression in a sample include DNA microarray, northern blotting and in situ hybridization (Parker & Barnes); RNAse protection assays (Hod), RT-PCR) (Weis et al.). Alternatively, antibodies can be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

Differential gene expression can also be identified, or confirmed using the DNA microarray technique. Thus, the expression profile of cancer-associated genes can be measured in either fresh or paraffin-embedded tumor tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of RNA typically is total RNA isolated from human tumors or tumor cell lines, and corresponding normal tissues or cell lines. Thus RNA can be isolated from a variety of primary tumors or tumor cell lines. If the source of RNA is a primary tumor, RNA can be extracted, for example, from frozen or archived paraffin-embedded and fixed (e.g. formalin-fixed) tissue samples, which are routinely prepared and preserved in everyday clinical practice.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a charge-coupled device (CCD) camera. Quantitation of hybridization of each arrayed element allows for assessment of corresponding RNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell (Schena et al.). Microarray analysis can be performed by commercially available equipment, following manufacturer's protocols, such as by using the Affymetrix GenChip technology, or Agilent's microarray technology.

The development of microarray methods for large-scale analysis of gene expression makes it possible to search systematically for molecular markers of cancer classification and outcome prediction in a variety of tumor types.

One of the most sensitive and most flexible quantitative PCR-based gene expression profiling methods is Reverse Transcriptase PCR (RT-PCR), which can be used to compare RNA levels in different sample populations, in normal and tumor tissues, with or without drug treatment, to characterize patterns of gene expression, to discriminate between closely related RNAs, and to analyze RNA structure.

The first step is the isolation of RNA/miR from a target sample. The starting material is typically total RNA isolated from human tumors or tumor cell lines, and corresponding normal tissues or cell lines, respectively. Thus RNA can be isolated from a variety of primary tumors, including breast, lung, colon, prostate, brain, liver, kidney, pancreas, spleen, thymus, testis, ovary, uterus, head and neck, etc., tumor, or tumor cell lines, with pooled DNA from donors. If the source of RNA is a primary tumor, RNA can be extracted, for example, from frozen or archived paraffin-embedded and fixed (e.g. formalin-fixed) tissue samples.

General methods for RNA extraction are well known in the art and are disclosed in standard textbooks of molecular biology, including Ausubel et al., Methods for RNA extraction from paraffin embedded tissues are disclosed, for example, in Rupp and Locker, and De Andres et al.). In particular, RNA isolation can be performed using purification kit, buffer set, and protease from commercial manufacturers, such as Qiagen, according to the manufacturer's instructions. For example, total RNA from cells in culture can be isolated using Qiagen RNeasy mini-columns or mirVana miRNA isolation kit (Applied Biosystems, California). Other commercially available RNA isolation kits include MASTERPURET™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN® (Applied Biosystems, California) PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a relatively constant level among different tissues, and is unaffected by the experimental treatment. RNAs frequently used to normalize patterns of gene expression are RNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorogenic probe (i.e., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al.

The steps of a representative protocol for profiling gene expression using fixed, paraffin-embedded (FFPE) tissues as the RNA source, including RNA isolation, purification, primer extension and amplification are given in various published journal articles, for example, Godfrey et al.; K. Specht et al. Briefly, a representative process starts with cutting about 10 μm thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by RT-PCR. RecoverAll (Ambion) is a kit for extracting all nucleic acid from FFPE tissues.

In the MassARRAY-based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor.

Further PCR-based techniques include, for example, differential display (Liang and Pardee); amplified fragment length polymorphism (iAFLP) (Kawamoto et al.); BEADARRAY™ technology (Illumina, San Diego, Calif.; Oliphant et al., Ferguson et al.); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al.); and high coverage expression profiling (HiCEP) analysis (Fukumura et al.).

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al.); and Velculescu et al.

Gene Expression Analysis by Massively Parallel Signature Sequencing (MPSS), as described by Brenner et al., Nature Biotechnology 18:630-634 (2000), is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

Immunohistochemistry methods are also suitable for detecting the expression levels of the prognostic markers of the present invention. Thus, antibodies or antisera, preferably polyclonal antisera, and most preferably monoclonal antibodies specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D polyacrylamide gel electrophoresis (2-D PAGE)); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

The present invention is surprisingly useful in predicting clinical outcome indicators in KRAS wildtype carcinomas, KRAS mutant carcinomas, and in carcinomas irrespective of KRAS status. Moreover, the present invention can be useful in combination with methods of assessing KRAS status. For example, the present invention includes KRAS mutational testing through biopsy of metastatic sites and allotment of tissue cores for both RNA and DNA purification. The high sensitivity and negative predictive value of the present invention cab be implemented to significantly enrich the responding patient population while minimizing the number of potential-responders (i.e. false negatives) who would be diverted from receiving useful therapy. Examples of KRAS analyses useful with the present invention are those developed by Canis Diagnostics, DxS (THERASCREEN®), Trimgen Corp (MUTECTOR TM II®), and Response Genetics.

The presently-disclosed subject matter includes devices and kits useful for predicting a clinical outcome indicator for a carcinoma of a subject. In some embodiments, a device is provided, comprising probes for detecting predictive markers. The device can be used to determine a profile of predictive markers, and can be used to practice the methods described herein above.

In some embodiments of the presently-disclosed subject matter a kit is provided for predicting a clinical outcome indicator for a carcinoma, including an algorithm for predicting a clinical outcome indicator for a carcinoma of a subject using levels of predictive markers in a biological sample. In some embodiments, the kit can comprise a program product (i.e., software product) for use in a computer device that executes program instructions recorded in a computer-readable medium to perform one or more steps of the methods described herein, such for estimating the efficacy of an EGFR inhibitor in treating a subject afflicted with cancer. In some embodiments, the kit can include a device comprising predictive marker probes.

The presently-disclosed subject matter is further illustrated by the following specific but non-limiting examples. The following examples may include compilations of data that are representative of data gathered at various times during the course of development and experimentation related to the present invention.

### EXAMPLES

### Example 1

### Selection of Exemplary Predictive Markers

Microarray analysis was performed on lung cancer cell lines (the “training set”) using the Affymetrix GeneChip Human Genome U133 platform (consisting of more than 22,000 sequences selected from GenBank®, dbEST, and RefSeq). The training set is a comparator set, i.e., a sample set used to establish the predictive algorithms of the presently-disclosed subject matter invention.

The lung cancer cell lines were analyzed for in vitro sensitivity to the EGFR-tyrosine kinase inhibitor erlotinib. Cell lines were designated as “resistant” or sensitive” based upon an empirically determined threshold.

The microarray data was compared with the erlotinib sensitivity status by using a significance analysis of microarrays (SAM) (e.g. as described by Tusher et al.).

The significance analysis identified 1495 useful sequences.

Using a Gene Ontology hierarchy program (e.g. as described by The Gene Ontology Consortium) to analyze the 1495 sequences identified above, 263 sequences were selected as being related to intracellular signaling (e.g., involving the EGF pathway).

Of the 263 sequences selected above, 180 sequences represented unique genes and were selected as Predictive Markers. The genes represented by these sequences are set forth in Table 1. The probe set identifiers are provided with reference to the most recently-available information as of the filing date of this application from Affymetrix, which is incorporated herein by this reference. The Gene IDs are provided with reference to the most recently-available information as of the filing date of this application from the National Center for Biotechnology Information (NCBI), which is incorporated herein by this reference. The P-values show the result of t-tests between ‘resistant’ and ‘sensitive’ cell lines (in vitro).

TABLE 1
Predictive Markers for Response to EGFR inhibitors

| Probe set | Gene | Description | p-value |
|---|---|---|---|
| 205891_at | ADORA2B | adenosine A2b receptor | 1.65E−12 |
| 213434_at | EPIM | epimorphin | 2.04E−12 |
| 211475_s_at | BAG1 | BCL2-associated athanogene | 1.21E−11 |
| 201716_at | SNX1 | sorting nexin 1 | 1.39E−11 |
| 219933_at | GLRX2 | glutaredoxin 2 | 2.82E−11 |
| 204513_s_at | ELMO1 | engulfment and cell motility 1 | 2.93E−11 |
| 203011_at | IMPA1 | inositol(myo)-1(or 4)-monophosphatase 1 | 4.20E−11 |
| 202743_at | PIK3R3 | phosphoinositide-3-kinase, regulatory | 4.52E−11 |
| | | subunit 3 (p55, gamma) | |
| 204491_at | PDE4D | Phosphodiesterase 4D, cAMP-specific | 8.05E−11 |
| 204000_at | GNB5 | guanine nucleotide binding protein (G | 8.77E−11 |
| | | protein), beta 5 | |
| 204115_at | GNG11 | guanine nucleotide binding protein (G | 1.03E−10 |
| | | protein), gamma 11 | |
| 218913_s_at | GMIP | GEM interacting protein | 2.64E−10 |
| 200994_at | IPO7 | importin 7 | 2.65E−10 |
| 202286_s_at | TACSTD2 | tumor-associated calcium signal | 2.75E−10 |
| | | transducer 2 | |
| 209035_at | MDK | midkine (neurite growth-promoting factor 2) | 7.32E−10 |
| 218995_s_at | EDN1 | endothelin 1 | 7.76E−10 |
| 219855_at | NUDT11 | nudix (nucleoside diphosphate linked | 8.78E−10 |
| | | moiety X)-type motif 11 | |
| 209678_s_at | PRKCI | protein kinase C, iota | 1.04E−09 |
| 202501_at | MAPRE2 | microtubule-associated protein, RP/EB | 2.31E−09 |
| | | family, member 2 | |
| 212117_at | RHOQ | ras homolog gene family, member Q | 3.22E−09 |
| 206277_at | P2RY2 | purinergic receptor P2Y, G-protein | 3.92E−09 |
| | | coupled, 2 | |
| 209295_at | TNFRSF10B | tumor necrosis factor receptor | 4.34E−09 |
| | | superfamily, member 10b | |
| 205376_at | INPP4B | inositol polyphosphate-4-phosphatase, | 4.51E−09 |
| | | type II, 105 kDa | |
| 206722_s_at | EDG4 | endothelial differentiation, | 7.97E−09 |
| | | lysophosphatidic acid GPCR, 4 | |
| 205673_s_at | ASB9 | ankyrin repeat and SOCS box-containing 9 | 1.25E−08 |
| 201471_s_at | SQSTM1 | sequestosome 1 | 1.34E−08 |
| 204352_at | TRAF5 | TNF receptor-associated factor 5 | 1.47E−08 |
| 206907_at | TNFSF9 | tumor necrosis factor (ligand) superfamily, | 1.58E−08 |
| | | member 9 | |
| 218150_at | ARL5 | ADP-ribosylation factor-like 5 | 2.05E−08 |
| 205459_s_at | NPAS2 | neuronal PAS domain protein 2 | 2.23E−08 |
| 205455_at | MST1R | macrophage stimulating 1 receptor (c-met- | 2.46E−08 |
| | | related tyrosine kinase) | |
| 202641_at | ARL3 | ADP-ribosylation factor-like 3 | 2.78E−08 |
| 201667_at | GJA1 | gap junction protein, alpha 1, 43 kDa | 2.86E−08 |
| | | (connexin 43) | |
| 210512_s_at | VEGF | vascular endothelial growth factor | 2.90E−08 |
| 212104_s_at | RBM9 | RNA binding motif protein 9 | 5.43E−08 |
| 200762_at | DPYSL2 | dihydropyrimidinase-like 2 | 5.43E−08 |
| 221235_s_at | TGFBRAP1 | transforming growth factor, beta receptor | 5.51E−08 |
| | | associated protein 1 | |
| 211302_s_at | PDE4B | phosphodiesterase 4B, cAMP-specific | 5.52E−08 |
| 205080_at | RARB | retinoic acid receptor, beta | 7.04E−08 |
| 202266_at | TTRAP | TRAF and TNF receptor associated | 7.29E−08 |
| | | protein | |
| 205240_at | GPSM2 | G-protein signalling modulator 2 (AGS3- | 8.31E−08 |
| | | like, C. elegans) | |
| 213798_s_at | CAP1 | CAP, adenylate cyclase-associated protein | 8.61E−08 |
| | | 1 (yeast) | |
| 221819_at | RAB35 | RAB35, member RAS oncogene family | 8.92E−08 |
| 207011_s_at | PTK7 | protein tyrosine kinase 7 | 9.79E−08 |
| 204255_s_at | VDR | vitamin D (1,25-dihydroxyvitamin D3) | 1.11E−07 |
| | | receptor | |
| 208864_s_at | TXN | thioredoxin | 1.34E−07 |
| 209885_at | RHOD | ras homolog gene family, member D | 1.50E−07 |
| 201923_at | PRDX4 | peroxiredoxin 4 | 1.61E−07 |
| 204392_at | CAMK1 | calcium/calmodulin-dependent protein | 2.24E−07 |
| | | kinase I | |
| 203269_at | NSMAF | neutral sphingomyelinase (N-SMase) | 2.59E−07 |
| | | activation associated factor | |
| 205924_at | RAB3B | RAB3B, member RAS oncogene family | 2.77E−07 |
| 202853_s_at | RYK | RYK receptor-like tyrosine kinase | 3.46E−07 |
| 202530_at | MAPK14 | mitogen-activated protein kinase 14 | 3.54E−07 |
| 219936_s_at | GPR87 | G protein-coupled receptor 87 | 4.07E−07 |
| 203665_at | HMOX1 | heme oxygenase (decycling) 1 | 4.38E−07 |
| 205926_at | IL27RA | interleukin 27 receptor, alpha | 5.33E−07 |
| 202105_at | IGBP1 | immunoglobulin (CD79A) binding protein 1 | 6.14E−07 |
| 213324_at | SRC | v-src sarcoma (Schmidt-Ruppin A-2) viral | 8.26E−07 |
| | | oncogene homolog (avian) | |
| 205709_s_at | CDS1 | CDP-diacylglycerol synthase | 8.69E−07 |
| | | (phosphatidate cytidylyltransferase) 1 | |
| 207303_at | PDE1C | phosphodiesterase 1C, calmodulin- | 9.11E−07 |
| | | dependent 70 kDa | |
| 204484_at | PIK3C2B | phosphoinositide-3-kinase, class 2, beta | 9.24E−07 |
| | | polypeptide | |
| 38269_at | PRKD2 | protein kinase D2 | 9.25E−07 |
| 211171_s_at | PDE10A | phosphodiesterase 10A | 1.06E−06 |
| 212757_s_at | CAMK2G | calcium/calmodulin-dependent protein | 1.09E−06 |
| | | kinase (CaM kinase) II gamma | |
| 202167_s_at | MMS19L | MMS19-like (MET18 homolog, | 1.19E−06 |
| | | S. cerevisiae) | |
| 202932_at | YES1 | v-yes-1 Yamaguchi sarcoma viral | 1.24E−06 |
| | | oncogene homolog 1 | |
| 209110_s_at | RGL2 | ral guanine nucleotide dissociation | 1.34E−06 |
| | | stimulator-like 2 | |
| 205055_at | ITGAE | integrin, alpha E | 1.48E−06 |
| 203910_at | PARG1 | PTPL1-associated RhoGAP 1 | 1.53E−06 |
| 203388_at | ARRB2 | arrestin, beta 2 | 1.59E−06 |
| 212099_at | RHOB | ras homolog gene family, member B | 1.77E−06 |
| 204497_at | ADCY9 | adenylate cyclase 9 | 1.79E−06 |
| 208091_s_at | DKFZP564K0822 | hypothetical protein DKFZp564K0822 | 1.81E−06 |
| 213135_at | TIAM1 | T-cell lymphoma invasion and metastasis 1 | 1.84E−06 |
| 203837_at | MAP3K5 | mitogen-activated protein kinase kinase | 1.89E−06 |
| | | kinase 5 | |
| 206549_at | INSL4 | insulin-like 4 (placenta) | 2.17E−06 |
| 205880_at | PRKD1 | protein kinase D1 | 2.3E−06 |
| 211471_s_at | RAB36 | RAB36, member RAS oncogene family | 2.3E−06 |
| 210058_at | MAPK13 | mitogen-activated protein kinase 13 | 2.44E−06 |
| 40850_at | FKBP8 | FK506 binding protein 8, 38 kDa | 2.88E−06 |
| 208819_at | RAB8A | RAB8A, member RAS oncogene family | 3.21E−06 |
| 202203_s_at | AMFR | autocrine motility factor receptor | 3.36E−06 |
| 201431_s_at | DPYSL3 | dihydropyrimidinase-like 3 | 3.54E−06 |
| 217976_s_at | DNCLI1 | dynein, cytoplasmic, light intermediate | 3.62E−06 |
| | | polypeptide 1 | |
| 201097_s_at | ARF4 | ADP-ribosylation factor 4 | 3.81E−06 |
| 203077_s_at | SMAD2 | SMAD, mothers against DPP homolog 2 | 3.82E−06 |
| | | (Drosophila) | |
| 203081_at | CTNNBIP1 | catenin, beta interacting protein 1 | 3.84E−06 |
| 219357_at | GTPBP1 | GTP binding protein 1 | 3.95E−06 |
| 212255_s_at | ATP2C1 | ATPase, Ca++ transporting, type 2C, | 4.41E−06 |
| | | member 1 | |
| 218360_at | RAB22A | RAB22A, member RAS oncogene family | 4.52E−06 |
| 202545_at | PRKCD | protein kinase C, delta | 4.62E−06 |
| 203679_at | TMED1 | transmembrane emp24 protein transport | 4.84E−06 |
| | | domain containing 1 | |
| 212070_at | GPR56 | G protein-coupled receptor 56 | 5.81E−06 |
| 204622_x_at | NR4A2 | nuclear receptor subfamily 4, group A, | 5.98E−06 |
| | | member 2 | |
| 202844_s_at | RALBP1 | ralA binding protein 1 | 6.56E−06 |
| 204547_at | RAB40B | RAB40B, member RAS oncogene family | 6.59E−06 |
| 219032_x_at | OPN3 | opsin 3 (encephalopsin, panopsin) | 7.09E−06 |
| 212422_at | PDCD11 | programmed cell death 11 | 7.2E−06 |
| 203266_s_at | MAP2K4 | mitogen-activated protein kinase kinase 4 | 7.43E−06 |
| 205220_at | GPR109B | G protein-coupled receptor 109B /// G | 7.59E−06 |
| | | protein-coupled receptor 109B | |
| 212181_s_at | NUDT4 | nudix (nucleoside diphosphate linked | 8.07E−06 |
| | | moiety X)-type motif 4 | |
| 208072_s_at | DGKD | diacylglycerol kinase, delta 130 kDa | 8.08E−06 |
| 218329_at | PRDM4 | PR domain containing 4 | 8.33E−06 |
| 210288_at | KLRG1 | killer cell lectin-like receptor subfamily G, | 8.46E−06 |
| | | member 1 | |
| 206099_at | PRKCH | protein kinase C, eta | 9.06E−06 |
| 205481_at | ADORA1 | adenosine A1 receptor | 1.03E−05 |
| 217839_at | TFG | TRK-fused gene | 1.04E−05 |
| 202340_x_at | NR4A1 | nuclear receptor subfamily 4, group A, | 1.12E−05 |
| | | member 1 | |
| 212873_at | HA-1 | minor histocompatibility antigen HA-1 | 1.25E−05 |
| 202020_s_at | LANCL1 | LanC lantibiotic synthetase component C- | 1.32E−05 |
| | | like 1 (bacterial) | |
| 209666_s_at | CHUK | conserved helix-loop-helix ubiquitous | 1.34E−05 |
| | | kinase | |
| 200651_at | GNB2L1 | guanine nucleotide binding protein (G | 1.41E−05 |
| | | protein), beta polypeptide 2-like 1 | |
| 201401_s_at | ADRBK1 | adrenergic, beta, receptor kinase 1 | 1.43E−05 |
| 203185_at | RASSF2 | Ras association (RalGDS/AF-6) domain | 1.52E−05 |
| | | family 2 | |
| 202401_s_at | SRF | serum response factor | 1.58E−05 |
| 203726_s_at | LAMA3 | laminin, alpha 3 | 1.63E−05 |
| 217496_s_at | IDE | insulin-degrading enzyme | 1.74E−05 |
| 206118_at | STAT4 | signal transducer and activator of | 1.8E−05 |
| | | transcription 4 | |
| 208641_s_at | RAC1 | ras-related C3 botulinum toxin substrate 1 | 1.93E−05 |
| 206044_s_at | BRAF | v-raf murine sarcoma viral oncogene | 1.98E−05 |
| | | homolog B1 | |
| 205349_at | GNA15 | guanine nucleotide binding protein (G | 2.02E−05 |
| | | protein), alpha 15 (Gq class) | |
| 1007_s_at | DDR1 | discoidin domain receptor family, member 1 | 2.04E−05 |
| 58994_at | FLJ20241 | putative NFkB activating protein | 2.13E−05 |
| 201895_at | ARAF | v-raf murine sarcoma 3611 viral oncogene | 2.13E−05 |
| | | homolog | |
| 211499_s_at | MAPK11 | mitogen-activated protein kinase 11 | 2.18E−05 |
| 203567_s_at | TRIM38 | tripartite motif-containing 38 | 2.23E−05 |
| 210621_s_at | RASA1 | RAS p21 protein activator (GTPase | 2.24E−05 |
| | | activating protein) 1 | |
| 219646_at | FLJ20186 | hypothetical protein FLJ20186 | 2.25E−05 |
| 218856_at | TNFRSF21 | tumor necrosis factor receptor | 2.29E−05 |
| | | superfamily, member 21 | |
| 205147_x_at | NCF4 | neutrophil cytosolic factor 4, 40 kDa | 2.44E−05 |
| 215177_s_at | ITGA6 | integrin, alpha 6 | 2.57E−05 |
| 202564_x_at | ARL2 | ADP-ribosylation factor-like 2 | 2.63E−05 |
| 207630_s_at | CREM | cAMP responsive element modulator | 2.73E−05 |
| 212629_s_at | PKN2 | protein kinase N2 | 2.8E−05 |
| 201181_at | GNAI3 | G protein alpha inhibiting activity | 2.87E−05 |
| | | polypeptide 3 | |
| 207375_s_at | IL15RA | interleukin 15 receptor, alpha | 2.91E−05 |
| 201983_s_at | EGFR | epidermal growth factor receptor | 2.95E−05 |
| 205263_at | BCL10 | B-cell CLL/lymphoma 10 | 3.07E−05 |
| 218186_at | RAB25 | RAB25, member RAS oncogene family | 3.17E−05 |
| 207111_at | EMR1 | egf-like module containing, mucin-like, | 3.4E−05 |
| | | hormone receptor-like 1 | |
| 219290_x_at | DAPP1 | dual adaptor of phosphotyrosine and 3- | 3.51E−05 |
| | | phosphoinositides | |
| 206456_at | GABRA5 | gamma-aminobutyric acid (GABA) A | 3.62E−05 |
| | | receptor, alpha 5 | |
| 219537_x_at | DLL3 | delta-like 3 (Drosophila) | 4.01E−05 |
| 200923_at | LGALS3BP | lectin, galactoside-binding, soluble, 3 | 4.18E−05 |
| | | binding protein | |
| 201390_s_at | CSNK2B | casein kinase 2, beta polypeptide | 4.29E−05 |
| 211992_at | WNK1 | WNK lysine deficient protein kinase 1 | 4.54E−05 |
| 205992_s_at | IL15 | interleukin 15 | 4.59E−05 |
| 200991_s_at | SNX17 | sorting nexin 17 | 4.73E−05 |
| 221610_s_at | STAP2 | signal-transducing adaptor protein-2 | 4.77E−05 |
| 201508_at | IGFBP4 | insulin-like growth factor binding protein 4 | 4.84E−05 |
| 219327_s_at | GPRC5C | G protein-coupled receptor, family C, | 6.06E−05 |
| | | group 5, member C | |
| 200985_s_at | CD59 | CD59 antigen p18-20 | 6.21E−05 |
| 202315_s_at | BCR | breakpoint cluster region | 6.46E−05 |
| 200627_at | TEBP | unactive progesterone receptor | 6.48E−05 |
| 201288_at | ARHGDIB | Rho GDP dissociation inhibitor (GDI) beta | 6.5E−05 |
| 205854_at | TULP3 | tubby like protein 3 | 7.09E−05 |
| 204369_at | PIK3CA | phosphoinositide-3-kinase, catalytic, alpha | 7.12E−05 |
| | | polypeptide | |
| 202012_s_at | EXT2 | exostoses (multiple) 2 | 7.25E−05 |
| 206204_at | GRB14 | growth factor receptor-bound protein 14 | 7.63E−05 |
| 201980_s_at | RSU1 | Ras suppressor protein 1 | 7.72E−05 |
| 210105_s_at | FYN | FYN oncogene related to SRC, FGR, YES | 7.73E−05 |
| 218589_at | P2RY5 | purinergic receptor P2Y, G-protein | 8.08E−05 |
| | | coupled, 5 | |
| 202150_s_at | NEDD9 | neural precursor cell expressed, | 8.37E−05 |
| | | developmentally down-regulated 9 | |
| 212273_x_at | GNAS | GNAS complex locus | 8.75E−05 |
| 200833_s_at | RAP1B | RAP1B, member of RAS oncogene family | 8.96E−05 |
| 214724_at | DIXDC1 | DIX domain containing 1 | 9.31E−05 |
| 207643_s_at | TNFRSF1A | tumor necrosis factor receptor | 9.57E−05 |
| | | superfamily, member 1A | |
| 219020_at | FLJ14249 | HS1-binding protein 3 | 9.74E−05 |
| 203895_at | PLCB4 | phospholipase C, beta 4 | 0.000101 |
| 204336_s_at | RGS19 | regulator of G-protein signalling 19 | 0.000107 |
| 217792_at | SNX5 | sorting nexin 5 | 0.000114 |
| 210056_at | RND1 | Rho family GTPase 1 | 0.000122 |
| 32137_at | JAG2 | jagged 2 | 0.000124 |
| 205596_s_at | SMURF2 | SMAD specific E3 ubiquitin protein ligase 2 | 0.000127 |
| 205698_s_at | MAP2K6 | mitogen-activated protein kinase kinase 6 | 0.000144 |
| 218931_at | RAB17 | RAB17, member RAS oncogene family | 0.000144 |
| 217763_s_at | RAB31 | RAB31, member RAS oncogene family | 0.00018 |
| 214875_x_at | APLP2 | amyloid beta (A4) precursor-like protein 2 | 0.000185 |
| 209184_s_at | IRS2 | insulin receptor substrate 2 | 0.000194 |
| 204602_at | DKK1 | dickkopf homolog 1 (Xenopus laevis) | 0.000261 |

### Example 2

### Predictive Markers in Colorectal Cancer

Over 100 CRC patients with colorectal cancer were biopsied from metastatic sites and the mutational status of KRAS was determined and gene expression data generated and reported Khambata-Ford et al.

These reported data were extracted in series matrix format from Gene Expression Omnibus (GEO) record GSE585112. The data from that study were scaled by the authors to mean intensity of 1500. Therefore, the data matrix was multiplied by a factor of 0.333 in order to reflect the mean intensity value of the data used to generate our predictive response (500). This direct linear relationship was confirmed by scaling experimental data to both values using Expression Console (Affymetrix, Santa Clara, Calif.) and observing the ratios on a probe by probe basis. A ratio of precisely 0.333 was observed for all probe sets, confirming the validity of this approach to data handling. The clinical response data and KRAS status were extracted from the supplementary files provided by Khambata-Ford et al.

The Predictive Markers set forth in Table 1 were imported into R for diagonal linear discriminant analysis (“DLDA”) according to Balko et al.

Data were separated into three datasets: KRAS wildtype, KRAS-mutant, or all patients combined. After sensitivity prediction on each of the datasets, the results were imported into Excel (Microsoft, Redmond, Wash.) and cross referenced with response and progression free survival (PFS).

Statistical Analysis.

All statistical analyses were performed using Prism (Graphpad, La Jolla, Calif.) and checked using JMP (SAS, Cary, N.C.). For comparisons of median progression free survival, the 2-tailed Mann-Whitney U test was performed between groups predicted to be sensitive and those predicted to be resistant. Kaplan-Meier survival curves were generated based on the PFS data reported by Khambata-Ford et al and analyzed by the log-rank statistic. These analyses were performed on only the KRAS-wildtype patient data first, and then repeated on all patient data as well as the KRAS-mutant population independently for comparison.

Results.

The gene expression profile of the Predictive Markers selected here predicts response and disease control of a cancer to EGFR inhibitors—in this example, cetuximab.

The microarray data from 80 of the 110 patients enrolled in that study were available for analysis. Of these, 43 (53.8%) were confirmed wildtype and 27 (33.8%) had confirmed KRAS mutations. The KRAS status of the remaining 10 (12.5%) patients was not reported.

When the prediction results were matched to response as reported by Khambata et al, the Predictive Markers of the present invention correctly captured 5/5 partial and complete KRAS wildtype responders to cetuximab. Also, 12/15 patients who demonstrated stable disease were classified as sensitive group by the Predictive Markers. Thus, the majority (17/20, 85%) of patients demonstrating overall disease control (SD+PR+CR) were captured in the ‘sensitive’ group.

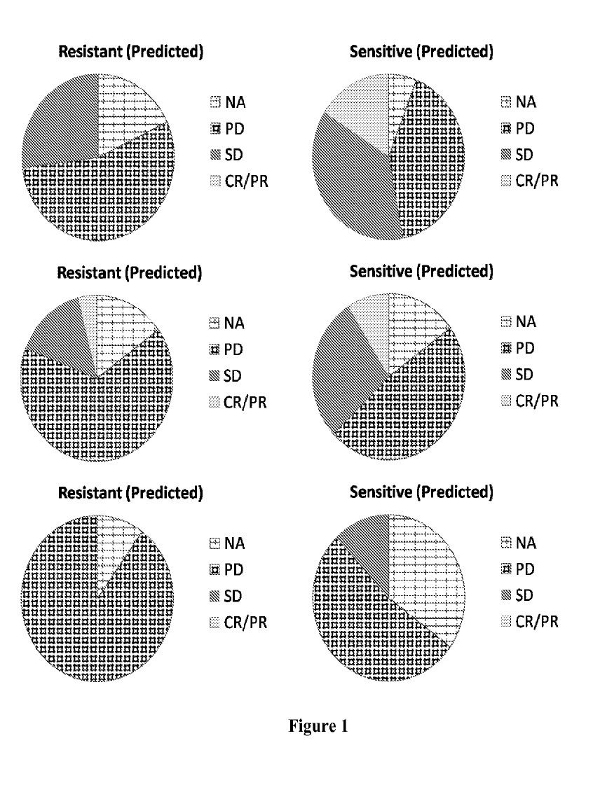

These results are shown in FIG. 1A where the following abbreviations apply: CR/PR—complete responses and partial responses; SD—stable disease; PD—progressive disease; NA—not available/not reported. Panel A shows KRAS-wildtype patients (43 subjects); Panel B shows all patients (80 subjects); and Panel C: shows KRAS-mutant patients (27 subjects).

We demonstrated that gene expression profile made from the Predictive Markers of the present invention is an independent predictor of response and/or disease control to EGFR inhibitors. This was demonstrated by analyzing all of the patient data, irrespective of KRAS status. Surprisingly, when all 80 patients were considered, the Predictive Markers of the present invention retained significant predictive capacity (FIG. 1B). Further surprising, 2/2 KRAS-mutant patients demonstrating stable disease to cetuximab were captured in the sensitive group (FIG. 1C). Calculated parameters (specificity, sensitivity, negative predictive value, and positive predictive value) for the ability of the model to predict disease control are given in Table 2.

TABLE 2
Calculated parameters for the ability of the
predictive model to predict disease control.

| Parameter | KRAS wildtype | All patients | KRAS mutant |
|---|---|---|---|
| Specificity | 0.32 | 0.4 | 0.5 |
| Sensitivity | 0.85 | 0.8 | 1 |
| PPV | 0.57 | 0.43 | 0.18 |
| NPV | 0.66 | 0.77 | 1 |

NPV: negative predictive value; PPV positive predictive value

### Example 3

### The Predictive Markers Stratify EGFR Inhibitor-Treated Carcinoma Patients Based on Progression-Free Survival

We next demonstrated that KRAS-wildtype patients identified by the model as ‘sensitive’ would exhibit true clinical benefit to treatment with cetuximab when compared to those classified as ‘resistant’.

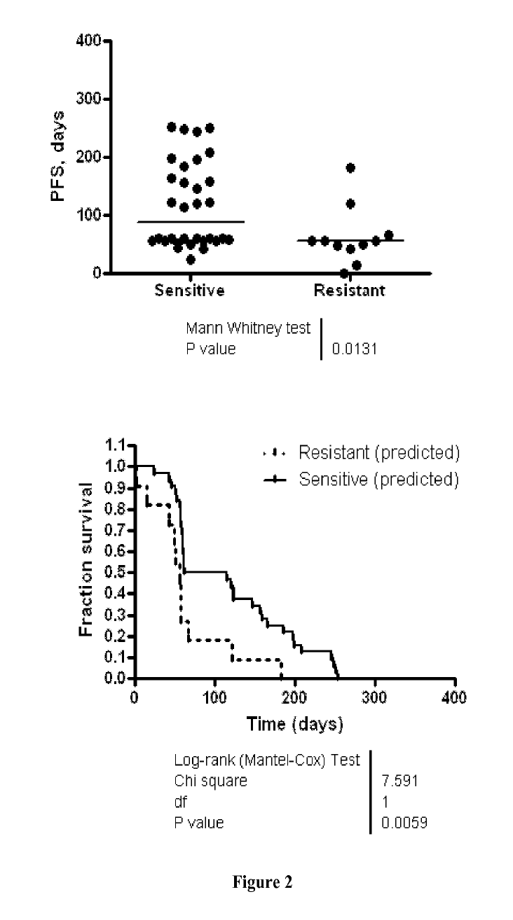

Scatter-plots and Kaplan-Meier survival curves of the progression-free survival (PFS) were generated for both predicted groups in KRAS-wildtype patients (FIG. 2). PFS was significantly greater in metastatic colorectal cancer (mCRC) KRAS-wildtype patients who predicted as ‘sensitive’ (median PFS: 88 days, mean PFS: 117 days, 95% CI: 90.8-143.8 days) compared to those that predicted as ‘resistant’ (median PFS: 56 days, mean PFS: 63 days, 95% CI: 29.9-96.9 days) (FIG. 2, left). The difference in PFS was statistically significant between groups (p=0.0133, two-tailed Mann-Whitney U test). The difference in the Kaplan-Meier survival curves was highly statistically significant when analyzed by the log-rank statistic for the KRAS-wildtype group (FIG. 2, right).

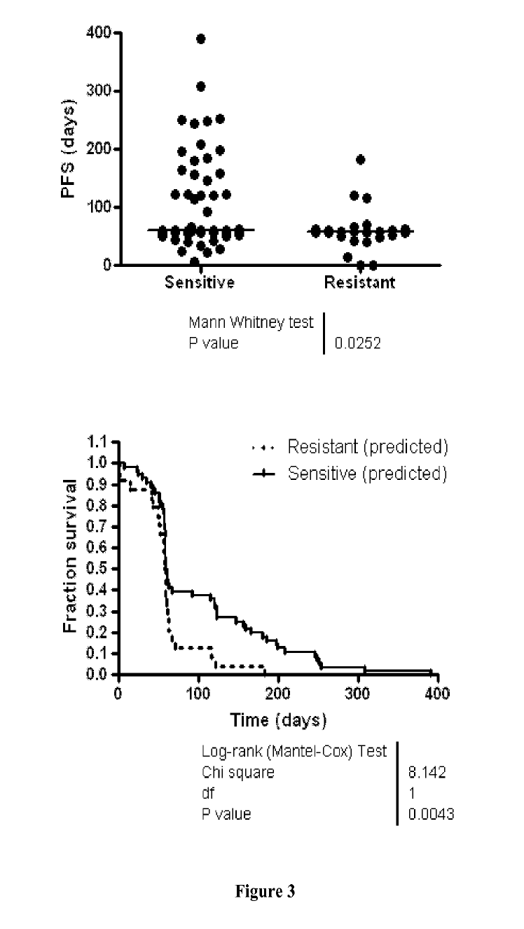

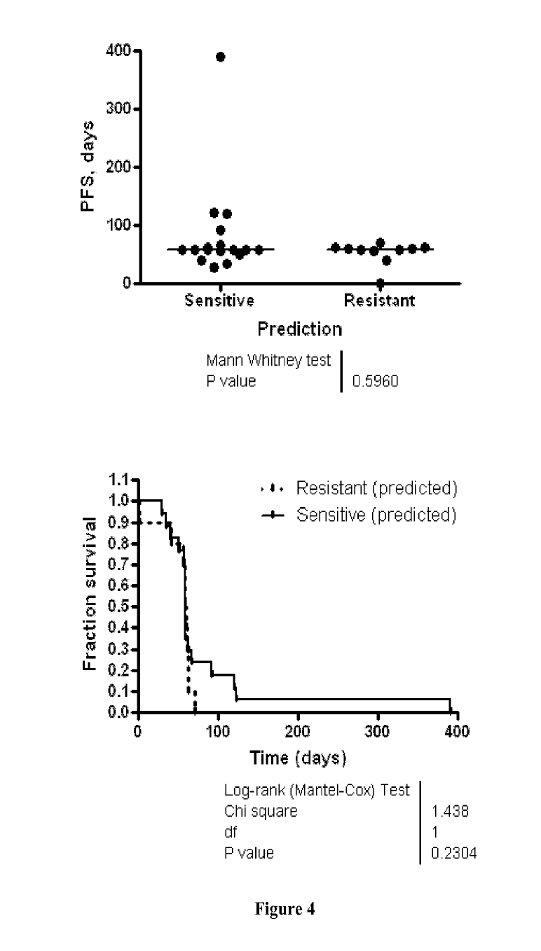

In FIG. 2 through FIG. 4, the scatter plots (left) depict the individual data points and median PFS for each group. The Kaplan-Meier survival curves (right) depict PFS between the ‘sensitive’ (green) and ‘resistant’ (red) groups. FIG. 2: KRAS-wildtype patients; FIG. 3: all patients; and FIG. 4: KRAS-mutant patients

When the entire cohort was included in the analysis, regardless of KRAS status, the difference remained statistically significant (p=0.0254, two-tailed Mann-Whitney U test) (FIG. 3, left). However, the differences in median and mean PFS were smaller (median PFS: 60 vs. 57.5 days and mean PFS: 104.7 vs. 60.5 days in ‘sensitive’ and ‘resistant’ subgroups, respectively). The difference in the Kaplan-Meier survival curves retained significance in the entire cohort, supporting use of Predictive Markers of the present invention as an independent predictor of EGFR inhibitor benefit (FIG. 3, right).

The results of analysis of KRAS mutant subjects are shown in FIG. 4.

EGFR inhibitors are frequently used in metastatic carcinomas and improve overall survival when used in unselected populations. However, a number of independent studies have elucidated the correlation of activating mutations in KRAS with lack of response to EGFR-targeted agents, and patient stratification based on KRAS status should improve overall survival through enrichment of responding patients. However, a significant number of KRAS-wildtype patients do not benefit from treatment, and therefore additional methods to enrich the treated population for responders were, until now, needed to reduce unnecessary toxicity and cost while maximizing therapeutic benefit from these agents.

In this study, we demonstrated that Predictive Markers of the present invention are useful for predicting response of carcinomas to EGFR inhibitors. It is surprising that the Predictive Markers have a high capacity to stratify KRAS-wildtype subjects who respond to treatment with EGFR inhibitors. The data were furthered by the significant separation of the survival curves of the predicted ‘sensitive’ group versus the predicted ‘resistant’ group.

We also demonstrated that Predictive Markers of the present invention are an independent predictor of EGFR inhibitor response. It is surprising that one patient with a KRAS-mutant tumor was reported by Khambata et al. to have had a PFS of >1 year on cetuximab, although radiographic response in this patient was not recorded. Our Predictive Markers classified this patient as ‘sensitive’ to cetuximab, offering additional support of the independency of our test from KRAS mutational status. Importantly, our methodology could easily be combined with KRAS mutational testing through biopsy of metastatic sites and allotment of tissue cores for both RNA and DNA purification. The high sensitivity and negative predictive value of the test supports the use of biomarkers of the present invention to significantly enrich the responding patient population while minimizing the number of potential-responders (i.e. false negatives) who would be diverted from receiving cetuximab.

In conclusion, these data demonstrate that Predictive Markers of the present invention are a valuable clinical tool in determining who should receive EGFR inhibitor therapy in carcinomas, and a valuable tool when used in combination with KRAS status.

Surprisingly, it has also been discovered that the present invention enables more precise stratification of carcinoma sensitivity, especially in KRAS wild type patients.

### Example 4

### Selecting Subsets of Predictive Markers for Lung Carcinoma

Of the Predictive Markers of Table 1, 176 Predictive Markers were selected based, in part, upon availability of TAQMAN assays. This set of 176 Predictive Markers (“PM Set A), does not contain the Predictive Markers of DDR1, TNFRSF21, GTPBP1, and LOC644617.

The gene expression profiles of the 42 non-small cell lung carcinoma cell lines (from above) using the PM Set A were analyzed. Genes were iteratively subtracted from the model and the analysis was re-performed to determine the usefulness of the subtracted gene. Based upon this method, a gene expression profile comprising 57 Predictive Markers was established as being especially useful (as set forth in Table 3).

TABLE 3
Subsets of Predictive Markers for Gene Expression Profiles.

| | | | | | | | | |
|---|---|---|---|---|---|---|---|---|
| | | Entrez | | | | | | |
| Probe Set ID | Gene Symbol | Gene ID | Gene Title | TAQMAN Assay | a | b | c | d |

a = Lung cancer subset 57; b = colorectal cancer subset - 26 genes;
c = colorectal cancer subset - 17 gene; d = colorectal cancer subset - 15 gene.

| | | | | | | | | |
|---|---|---|---|---|---|---|---|---|
| 1007_s_at | DDR1 | 780 | discoidin domain receptor | | | X | | |
| | | | tyrosine kinase 1 | | | | | |
| 200762_at | DPYSL2 | 1808 | dihydropyrimidinase-like 2 | Hs00954558_m1 | X | | | |
| 200994_at | IPO7 | 10527 | Importin 7 | Hs01020544_m1 | X | | | |
| 201471_s_at | SQSTM1 | 8878 | sequestosome 1 | Hs01061917_g1 | X | | | |
| 201667_at | GJA1 | 2697 | gap junction protein, alpha 1, | Hs00748445_s1 | X | | | |
| | | | 43 kDa | | | | | |
| 201716_at | SNX1 | 6642 | sorting nexin 1 | Hs00541726_m1 | X | | | |
| 201923_at | PRDX4 | 10549 | peroxiredoxin 4 | Hs01056078_m1 | X | X | X | X |
| 202105_at | IGBP1 | 3476 | immunoglobulin (CD79A) | Hs00426831_mH | X | | | |
| | | | binding protein 1 | | | | | |
| 202266_at | TTRAP | 51567 | TTRAF and TNF receptor | Hs00213282_m1 | X | | | |
| | | | associated protein | | | | | |
| 202286_s_at | TACSTD2 | 4070 | tumor-associated calcium signal | Hs00242741_s1 | X | | | |
| | | | transducer 2 | | | | | |
| 202501_at | MAPRE2 | 10982 | microtubule-associated protein, | Hs00936741_m1 | X | | | |
| | | | RP/EB family, member 2 | | | | | |
| 202530_at | MAPK14 | 1432 | mitogen-activated protein kinase | Hs01047704_m1 | X | | | |
| | | | 14 | | | | | |
| 202641_at | ARL3 | 403 | ADP-ribosylation factor-like 3 | Hs00188824_m1 | X | | | |
| 202743_at | PIK3R3 | 8503 | phosphoinositide-3-kinase, | Hs00300461_s1 | X | | | |
| | | | regulatory subunit 3 (gamma) | | | | | |
| 202853_s_at | RYK | 6259 | RYK receptor-like tyrosine | Hs01070538_g1 | X | X | X | X |
| | | | kinase | | | | | |
| 203011_at | IMPA1 | 3612 | inositol(myo)-1(or 4)- | Hs00183279_m1 | X | | | |
| | | | monophosphatase 1 | | | | | |
| 203269_at | NSMAF | 8439 | neutral sphingomyelinase (N- | Hs01060817_m1 | X | | | |
| | | | SMase) activation associated | | | | | |
| | | | factor | | | | | |
| 203665_at | HMOX1 | 3162 | heme oxygenase (decycling) 1 | Hs01110251_m1 | X | X | X | X |
| 204000_at | GNB5 | 10681 | guanine nucleotide binding | Hs01062967_m1 | X | X | X | X |
| | | | protein (G protein), beta 5 | | | | | |
| 204115_at | GNG11 | 2791 | guanine nucleotide binding | Hs00914578_m1 | X | | | |
| | | | protein (G protein), gamma 11 | | | | | |
| 204255_s_at | VDR | 7421 | vitamin D (1,25- | Hs01045840_m1 | X | | | |
| | | | dihydroxyvitamin D3) receptor | | | | | |
| 204352_at | TRAF5 | 7188 | TNF receptor-associated factor 5 | Hs01072217_m1 | X | | | |
| 204369_at | PIK3CA | 5290 | phosphoinositide-3-kinase, | Hs00907957_m1 | | X | | |
| | | | catalytic, alpha polypeptide | | | | | |
| 204392_at | CAMK1 | 8536 | calcium/calmodulin-dependent | Hs01114209_g1 | X | | | |
| | | | protein kinase I | | | | | |
| 204491_at | PDE4D | 5144 | phosphodiesterase 4D, cAMP- | Hs01579629_g1 | X | | | |
| | | | specific (phosphodiesterase E3 | | | | | |
| | | | dunce homolog, Drosophila) | | | | | |
| 204513_s_at | ELMO1 | 9844 | engulfment and cell motility 1 | Hs00404994_m1 | X | X | X | X |
| 205080_at | RARB | 5915 | retinoic acid receptor, beta | Hs00977142_mH | X | | | |
| 205240_at | GPSM2 | 29899 | G-protein signaling modulator 2 | Hs01076888_m1 | X | X | X | X |
| | | | (AGS3-like, C. elegans) | | | | | |
| 205376_at | INPP4B | 8821 | inositol polyphosphate-4- | Hs01038089_m1 | X | | | |
| | | | phosphatase, type II, 105 kDa | | | | | |
| 205455_at | MST1R | 4486 | macrophage stimulating 1 | Hs00899925_m1 | X | | | |
| | | | receptor (c-met-related tyrosine | | | | | |
| | | | kinase) | | | | | |
| 205459_s_at | NPAS2 | 4862 | neuronal PAS domain protein 2 | Hs01551850_m1 | X | | | |
| 205673_s_at | ASB9 | 140462 | ankyrin repeat and SOCS box- | Hs00932804_m1 | X | | | |
| | | | containing 9 | | | | | |
| 205709_s_at | CDS1 | 1040 | CDP-diacylglycerol synthase | Hs00996217_mH | X | | | |
| | | | (phosphatidate | | | | | |
| | | | cytidylyltransferase) 1 | | | | | |
| 205891_at | ADORA2B | 136 | adenosine A2b receptor | Hs00386497_m1 | X | | | |
| 205924_at | RAB3B | 5865 | RAB3B, member RAS oncogene | Hs01001137_m1 | X | | | |
| | | | family | | | | | |
| 205926_at | IL27RA | 9466 | interleukin 27 receptor, alpha | Hs00945027_g1 | X | | | |
| 206277_at | P2RY2 | 5029 | purinergic receptor P2Y, G- | Hs01923024_s1 | X | | | |
| | | | protein coupled, 2 | | | | | |
| 206722_s_at | LPAR2 | 9170 | lysophosphatidic acid receptor 2 | Hs00173704_m1 | X | | | |
| 206907_at | TNFSF9 | 8744 | tumor necrosis factor (ligand) | Hs00355396_m1 | X | | | |
| | | | superfamily, member 9 | | | | | |
| 207011_s_at | PTK7 | 5754 | PTK7 protein tyrosine kinase 7 | Hs00897151_m1 | X | X | X | X |
| 207643_s_at | TNFRSF1A | 7132 | tumor necrosis factor receptor | Hs00533568_g1 | | X | | |
| | | | superfamily, member 1A | | | | | |
| 208091_s_at | ECOP | 81552 | EGFR-coamplified and | Hs01033978_m1 | | X | X | X |
| | | | overexpressed protein | | | | | |
| 208641_s_at | RAC1 | 5879 | ras-related C3 botulinum toxin | Hs01588892_g1 | | X | | |
| | | | substrate 1 (rho family, small | | | | | |
| | | | GTP binding protein Rac1) | | | | | |
| 208864_s_at | TXN | 7295 | thioredoxin | Hs01555214_g1 | X | | | |
| 209035_at | MDK | 4192 | midkine (neurite growth- | Hs00171064_m1 | X | | | |
| | | | promoting factor 2) | | | | | |
| 209110_s_at | RGL2 | 5863 | ral guanine nucleotide | Hs01588050_g1 | | X | X | X |
| | | | dissociation stimulator-like 2 | | | | | |
| 209295_at | TNFRSF10B | 8795 | tumor necrosis factor receptor | Hs00366278_m1 | X | X | X | X |
| | | | superfamily, member 10b | | | | | |
| 209678_s_at | PRKCI | 5584 | protein kinase C, iota | Hs00995849_g1 | X | X | X | X |
| 209885_at | RHOD | 29984 | ras homolog gene family, | Hs00989684_m1 | X | | | |
| | | | member D | | | | | |
| 210058_at | MAPK13 | 5603 | mitogen-activated protein kinase | Hs00559620_g1 | | X | X | |
| | | | 13 | | | | | |
| 210512_s_at | VEGFA | 7422 | vascular endothelial growth factor A | Hs00900058_m1 | X | X | X | X |
| 211302_s_at | PDE4B | 5142 | phosphodiesterase 4B, cAMP- | Hs00277080_m1 | X | | | |
| | | | specific (phosphodiesterase E4 | | | | | |
| | | | dunce homolog, Drosophila) | | | | | |
| 211475_s_at | BAG1 | 573 | BCL2-associated athanogene | Hs01105460_g1 | X | | | |
| 212099_at | RHOB | 388 | ras homolog gene family, | Hs00269660_s1 | | X | X | X |
| | | | member B | | | | | |
| 212104_s_at | RBM9 | 23543 | RNA binding motif protein 9 | Hs00329214_s1 | X | | | |
| 212117_at | RHOQ | 23433 | ras homolog gene family, | Hs00865365_s1 | X | | | |
| | | | member Q | | | | | |
| 212181_s_at | NUDT4 /// | 11163 /// | nudix (nucleoside diphosphate | Hs01903319_s1 | | X | | |
| | NUDT4P1 | 440672 | linked moiety X)-type motif 4 /// | | | | | |
| | | | nudix (nucleoside diphosphate | | | | | |
| | | | linked moiety X)-type motif 4 | | | | | |
| | | | pseudogene 1 | | | | | |
| 212255_s_at | ATP2C1 | 27032 | ATPase, Ca++ transporting, type | Hs00995940_m1 | | X | X | |
| | | | 2C, member 1 | | | | | |
| 212273_x_at | GNAS | 2778 | GNAS complex locus | Hs00894277_g1 | | X | | |
| 212757_s_at | CAMK2G | 818 | calcium/calmodulin-dependent | Hs00538470_g1 | | X | X | X |
| | | | protein kinase (CaM kinase) II | | | | | |
| | | | gamma | | | | | |
| 213324_at | SRC | 6714 | v-src sarcoma (Schmidt-Ruppin | Hs01082239_g1 | X | | | |
| | | | A-2) viral oncogene homolog | | | | | |
| | | | (avian) | | | | | |
| 213434_at | STX2 | 2054 | syntaxin 2 | Hs00181827_m1 | X | | | |
| 214724_at | DIXDC1 | 85458 | DIX domain containing 1 | Hs00736707_m1 | X | | | |
| 215177_s_at | ITGA6 | 3655 | integrin, alpha 6 | Hs01041013_m1 | | X | | |
| 218186_at | RAB25 | 57111 | RAB25, member RAS oncogene | Hs00220628_m1 | X | | | |
| | | | family | | | | | |
| 218589_at | P2RY5 | 10161 | purinergic receptor P2Y, G- | Hs00271758_s1 | | X | X | X |
| | | | protein coupled, 5 | | | | | |
| 218931_at | RAB17 | 64284 | RAB17, member RAS oncogene | Hs00940835_g1 | X | | | |
| | | | family | | | | | |
| 219020_at | HS1BP3 | 64342 | HCLS1 binding protein 3 | Hs00372728_m1 | X | | | |
| 219933_at | GLRX2 | 51022 | glutaredoxin 2 | Hs01567400_m1 | X | | | |
| 219936_s_at | GPR87 | 53836 | G protein-coupled receptor 87 | Hs00225057_m1 | X | | | |
| 221610_s_at | STAP2 | 55620 | signal transducing adaptor family | Hs00214588_m1 | X | | | |
| | | | member 2 | | | | | |
| 38269_at | PRKD2 | 25865 | protein kinase D2 | Hs01041279_g1 | | X | | |
| 58994_at | CC2D1A | 54862 | coiled-coil and C2 domain | Hs01089703_g1 | | X | | |
| | | | containing 1A | | | | | |

### Example 5

### Selecting Subsets of Predictive Markers for Colorectal Cancer

The gene expression profile of biopsies from colorectal cancer using the PM Set A were analyzed for correlation with progression free survival of the subjects from which the cancer biopsies were derived. Predictive Markers that were more highly expressed in the sensitive NSCLC cell lines relative to the resistant lines while also being more highly expressed in CRC patients with a PFS of >150 days were retained. Similarly, genes that were more highly expressed in the resistant NSCLC cell lines relative to the sensitive cell lines while also being more highly expressed in CRC patients with a PFS of <50 days were retained. Finally, correlations of the remaining genes with PFS were calculated and those with an absolute value of correlation >0.2 were retained while those that were <0.2 were filtered. Based upon this correlation, a gene expression profile comprising 26 Predictive Markers (set forth in Table 3) was established as being especially useful.

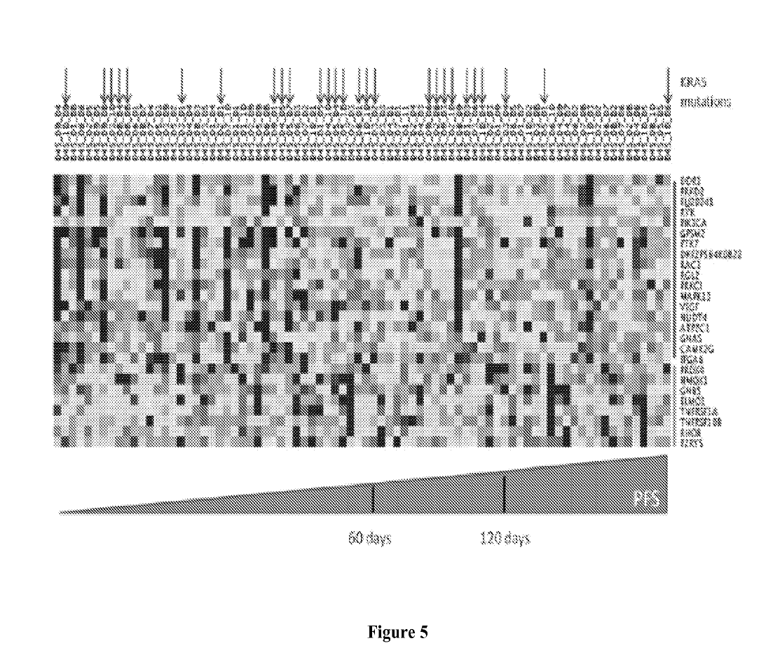

A heatmap of the signal intensities of the 26 Predictive Marker set demonstrated a pattern of deregulation coincident with PFS as shown in FIG. 5. All clinical samples are included (KRAS WT, KRAS-mutant, and unconfirmed/NA) and are arranged according to PFS. KRAS-mutant samples (codon 12) are designated by red arrows.

Moreover, this 26 Predictive Marker set further improved stratification of PFS over the 180 Predictive Marker set. Calculated parameters (specificity, sensitivity, negative predictive value, and positive predictive value) for the ability of the model to predict disease control are given in Table 4.

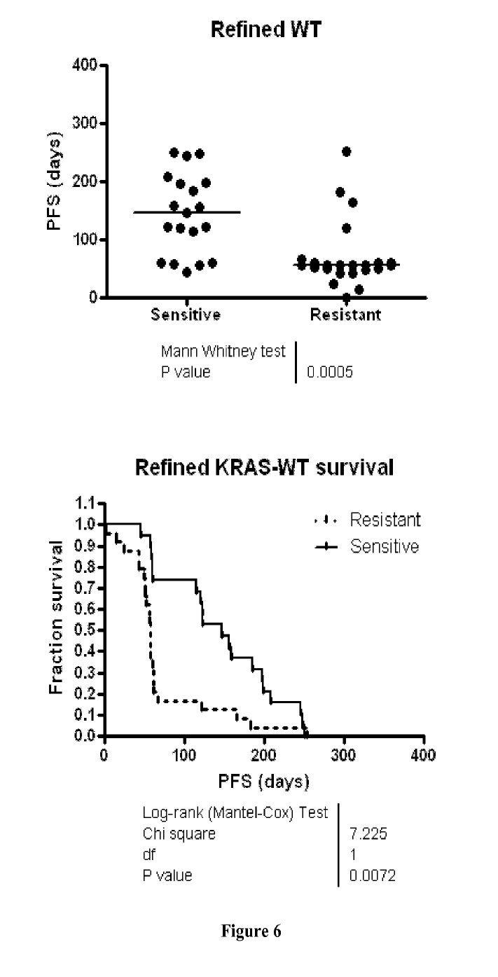

When the 26 Predictive Marker set algorithm was applied to the KRAS-WT CRC data, the resulting difference in PFS between the predicted-sensitive and predicted-resistant group was highly significant. As shown in FIG. 6B, the confirmed KRAS-WT samples were plotted by predicted sensitivity. Closed circles and bars represent individual PFS and median PFS for the group, respectively. Kaplan-Meier curves demonstrated significant separation as well. Parameters (sensitivity, specificity, NPV and PPV) for the refined model to predict disease control (SD/PR/CR) are presented in Table 3.

The Kaplan-Meier curves (FIG. 6C) demonstrated significant separation of PFS between the ‘sensitive’ (green) and ‘resistant’ (red) groups. The reported p-value is for the log-rank statistic.

TABLE 4
Calculated parameters for the ability 26 Predictive Marker
gene expression profile to predict disease control.

| Parameter | KRAS-wildtype | All patients | KRAS-mutant |
|---|---|---|---|
| Specificity | 0.74 | 0.71 | 0.78 |
| Sensitivity | 0.80 | 0.80 | 1.00 |
| PPV | 0.76 | 0.63 | 0.33 |
| NPV | 0.78 | 0.86 | 1.00 |

### Example 6

### Selecting Further Subsets of Predictive Markers for Colorectal Cancer

The gene expression profile of the 26 sequences selected above was analyzed in order to determine whether any sequences can be eliminated and still retain substantial usefulness in predicting clinical outcome. By subtracting one gene at a time and analyzing the gene expression profile for correlation with progression-free survival, two especially useful subsets were identified, namely a 17 and a 15 Predictive Marker subset as set forth in Table 3.

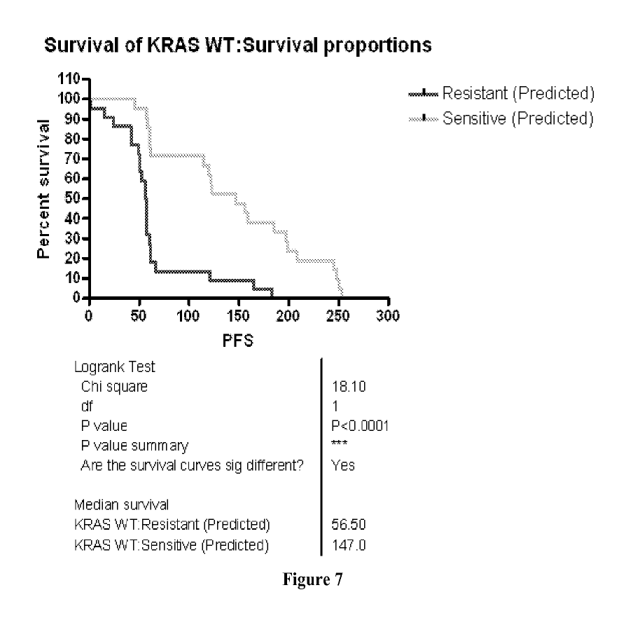

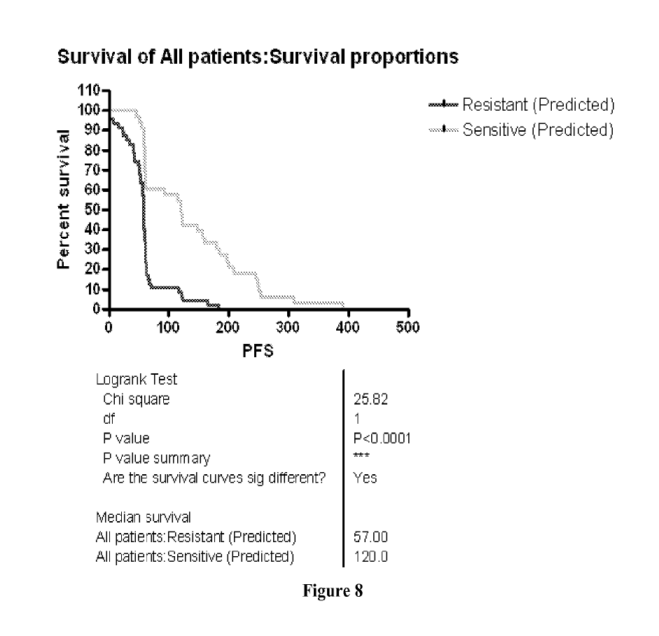

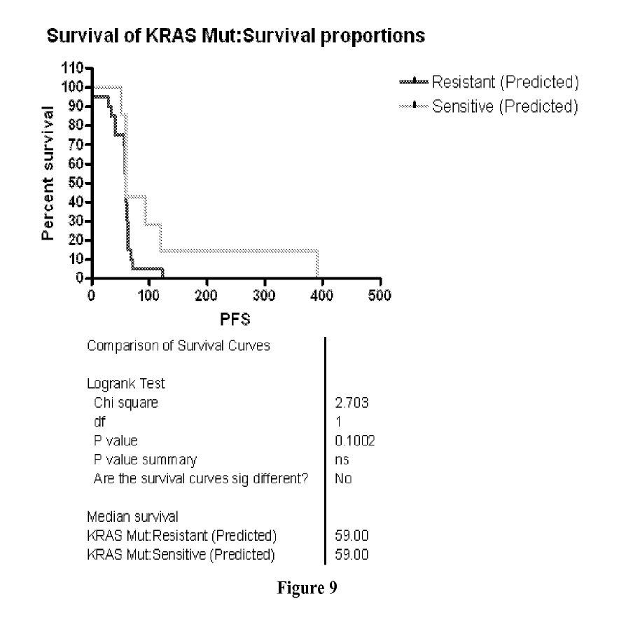

We surprisingly found that using a relatively small set of Predictive Markers of the present invention selected by methods taught herein, yielded greater differences in median survival between the predicted ‘resistant’ and ‘sensitive’ groups as well as greatly improving the sensitivity and specificity of the assay to greater than 75% each. As shown in FIG. 7, 15 Predictive markers, stratified KRAS positive CRC patients for survival according to EGFR inhibitor sensitivity. Similar results were obtained using the 15 Predictive Markers, stratifying all CRC patients (FIG. 8) and KRAS negative CRC patients (FIG. 9).

### Example 7

### Predictive Markers for Pancreatic Cancer

The gene expression profiles of the 180, 176, 26, 17, and 15 Predictive Marker sets are evaluated for value in predicting EGFR inhibitor sensitivity and profile in pancreatic cancer. Using tissue from more than 10 different cancers, results show a positive correlation statistical correlation between predicted sensitivity and observed sensitivity and between predicted survival and observed survival in each of the 5 Predictive Marker sets.

### Example 8

### Predictive Markers for Head and Neck Cancers

The gene expression profiles of the 180, 176, 26, 17, and 15 Predictive Marker sets are evaluated for value in predicting EGFR inhibitor sensitivity and profile in head and neck cancers. Using tissue from more than 10 different cancers, results show a positive correlation statistical correlation between predicted sensitivity and observed sensitivity and between predicted survival and observed survival in each of the 5 Predictive Marker sets.

### Example 9

### Exemplary miR Predictive Markers

Recently, regulation of gene expression by the action of microRNAs has garnered significant attention in the field of cancer biology. MicroRNAs are small, non-coding fragments of RNA that are localized in the cytoplasm of cells. There, these small RNAs bind to complimentary sequences on mRNA and enucleate them into RNA-induced silencing complexes (RISC) (Filipowicz et al., 2008). The RISC may inhibit translation by preventing the translational machinery from binding the RNA or may sentence the bound mRNA to degradation. microRNA genes are annotated as families and each family inhibits protein expression from a number of messages. The expression of many microRNAs is deregulated in tumors as compared to normal tissue, and therefore, microRNAs may function as either tumor suppressors or oncogenes, depending on the mRNA they target (Lu et al., 2005; Wiemer, 2007).

As described herein, a profile of microRNA (miR) expression values can be used to predict a clinical outcome indicator for a carcinoma, including EGFR inhibitor sensitivity or survival. A microRNA profile including miRs as set forth in Table 5 can predict sensitivity of a cancer to treatment with EGFR inhibitors.

TABLE 5
miR Predictive Markers for Response to EGFR inhibitors.

miR-135b
miR-140-3p
miR-141
miR-197
miR-200b
miR-200c
miR-205
miR-224
miR-301a
miR-34c-5p
miR-518f
miR-628-5p
miR-636

It is described in this example that a miRNA expression profile can be used to predict sensitivity to erlotinib using NSCLC and PDAC cell lines can be used to elucidate additional biomarkers to predict sensitivity to EGFR inhibition. Treatment with inhibitors of the epidermal growth factor receptor (EGFR) can result in clinical response in both non-small cell lung cancer (NSCLC) and pancreatic ductal adenocarcinoma (PDAC), but only in a minority of unselected patients. Identification of markers that can direct treatment to the responsive population is described herein, specifically, described in this example is the generation of a microRNA profile of response to EGFR inhibition.

Using a TAQMAN MicroRNA Array of 381 probes, we demonstrate that a multi-microRNA profile derived from NSCLC cell lines can predict sensitivity of both lung and pancreatic cancer cell lines to erlotinib, a small molecule inhibitor of EGFR.

NSCLC cell lines A549, UKY-29, H460, H1975, H358, H1650, PC-9, and H3255, H322, H820, HCC 827, H2122 and pancreatic cell lines MiaPaca, Panel, Aspcl, and BxPc3 (panel of cell lines) were analyzed for differential miRNA expression using TAQMAN miRNA arrays. For each cell line, total RNA was isolated followed by cDNA preparation and preamplification. Real-time PCR and hybridization to the TAQMAN MicroRNA Array. A panel of 381 miRNA probes generated CT values for analysis.

The panel of NSCLC cell lines were separated into sensitive and resistant to erlotinib treatment using an apoptosis assay after 48 h as a measure (Balko et al., 2006). Resistant cell lines (A549, UKY-29, H460, and H1975) and sensitive cell lines (H1650, H3255, PC-9, and H358) are used to evaluate differential microRNA expression. All cell lines were passaged as previously published and harvested after 2 days growth in serum-containing media (Balko et al., 2006). Lung cancer cell lines sensitive to erlotinib (H322 and HCC827) and resistant to erlotinib (H2122 and H820) and pancreatic cancer cell lines sensitive to erlotinib (Aspc-1 and Bxpc-3) and resistant to erlotinib (Mia PaCa-2 and Panc-1) were used for validation of the predictor, as previously described, again harvested after 2 days growth in serum-containing media (Tzeng et al., 2007).

RNA was prepared for cDNA synthesis. The cDNA was pre-amplified as described by ABI before qRT-PCR and array hybridization. The gene expression data were acquired following hybridization to the TAQMAN arrays, normalized, and filtered prior to the application of a t-test to determine genes significantly-deregulated between erlotinib sensitive and resistant cell lines. The resulting genes were also analyzed for differences in their expression levels and gene microRNA profiles were obtained.

The microRNA profile of response to erlotinib in NSCLC cell lines was imported into the previously published diagonal linear discriminant model to train a predictive model of sensitivity to erlotinib (Balko et al., 2006). Expression data for four additional NSCLC cell lines and the four pancreatic cancer cell lines were used as a validation set for prediction. RNA from eight tumors was also used for validation of predictor lung and colorectal cancers. It is found that the profile is capable of predicting sensitivity of these cell lines to erlotinib.

Also examined was the biological contribution of the deregulated microRNAs in both the NSCLC and pancreatic cancer cell lines to erlotinib sensitivity.

The microRNA profile of response described herein using the panel of NSCLC cell lines suggests that genes responsible for controlling EMT are important for predicting sensitivity to erlotinib. Therefore, the present inventor returned to the previously published gene expression data to evaluate other genes responsible for control of EMT. It was found that the transcription factor ZEB1 (TCF8) was re down-regulated in erlotinib-sensitive NSCLC.

The multi-miRNA profile of sensitivity provides a means for defining response that can be generated from FFPE samples of tumor rather than fresh tumor samples. MicroRNA can be isolated from FFPE samples, surviving the fixation process, without risk of degradation associated with longer RNAs. Thus, response to EGFR inhibition in the second- or third-line can be predicted from fixed tumor samples collected early in the treatment of an individual patient, eliminating the need for acquisition of fresh tumor. Further, the microRNA profile identifies the well-studied EMT pathway indicative of EGFR-inhibitor sensitivity and provides biological significance for the members of the profile.

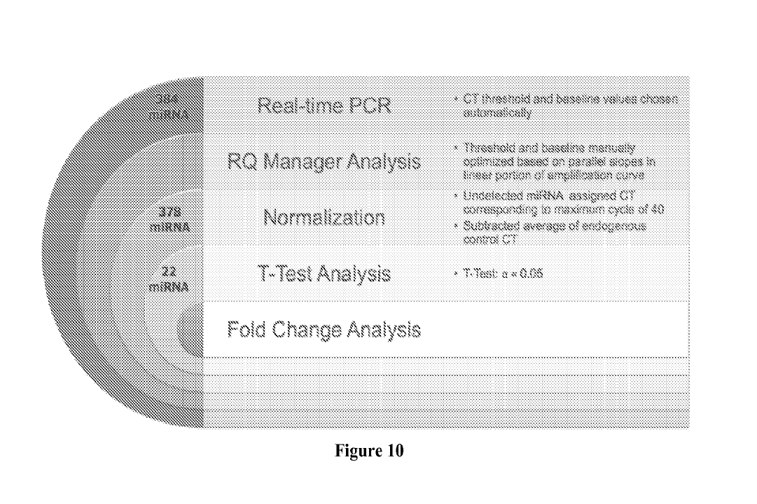

With reference to FIG. 10, data from real-time PCR was analyzed using RQ Manager software. CT values of 4 sensitive (H1650, H3255, H358, and PC-9) and 4 resistant (A549, UKY-29, H460, and H1975) lines were assessed for significance via t-test with α=0.05. Significant miRs were used to build a predictive profile and diagonal linear discriminant analysis (DLDA) was carried out using H322, H820, H2122 and HCC827 and the four pancreatic cell lines as validation.

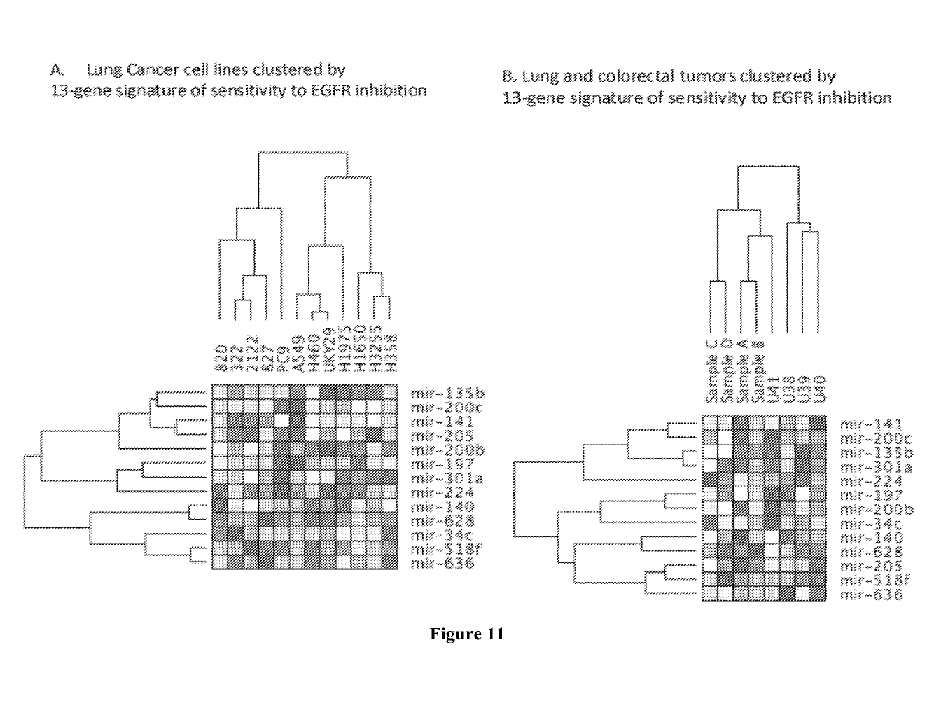

With reference to FIG. 11, hierarchical clustering of expression data from 13 differentially-expressed miRNA identified from lung cancer cell lines with differential sensitivity to erlotinib (left) and tumors (right). Darker shades are highly expressed while lighter shades are expressed at low levels.

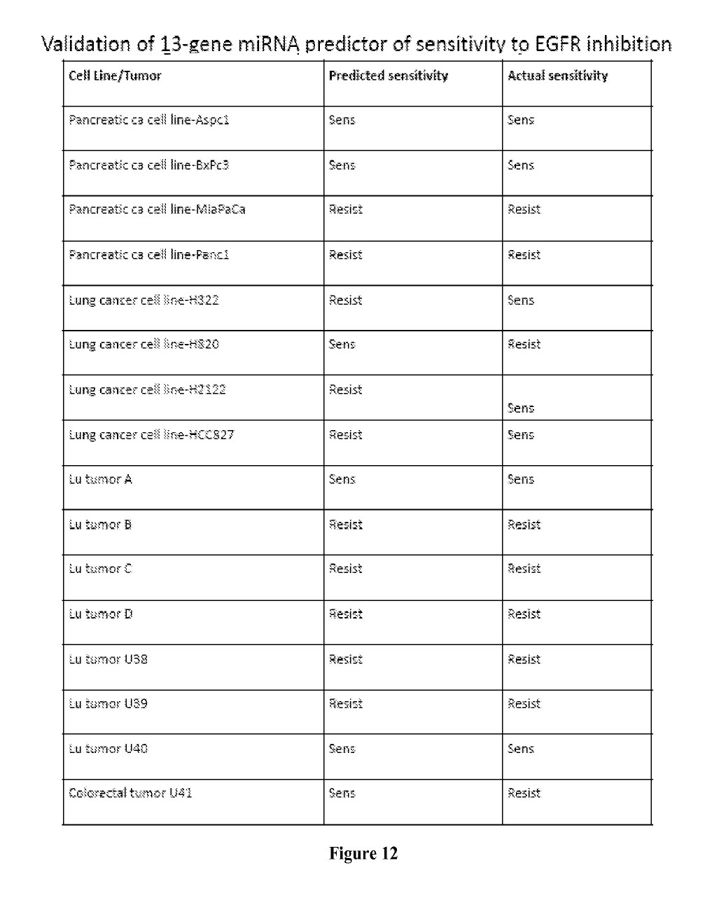

With reference to FIG. 12, DLDA prediction of sensitivity to erlotinib in pancreatic cancer cell lines (top) and lung tumors (bottom) is generated. Lung cancer cell line data (B) were used to train the predictor for sensitivity to erlotinib. DLDA was performed on the cell lines and tumors using RT-PCR data from 13 of the miRNA and assigned as core of sensitivity and resistance (FIG. 12). {Resist} or {Sens} denotes observed and predicted sensitive or resistant phenotype to EGFR inhibitor treatment.

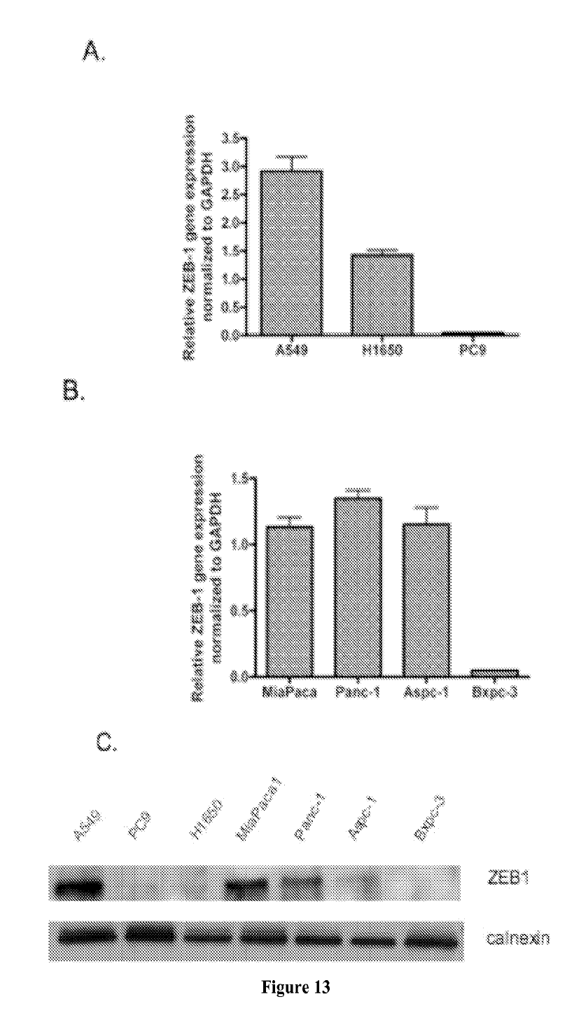

With reference to FIG. 13, overlap of the differentially-expressed miRs, between lung and pancreatic cancer cell lines, demonstrates alteration in miRNA targeting EMT genes. Are presentative target, ZEB1, was analyzed in both lung and pancreatic cancer cell lines form RNA expression (A and B, above) and protein expression (C).

These data demonstrate that sensitivity to an EGFR inhibitor can be predicted by multi-gene microRNA profiles for various cancer-types.

The microRNA profile of response of sensitivity to erlotinib was different for NSCLC and pancreatic cancer cell lines, but contained considerable concordance. ZEB1 was chosen to pursue as it is a transcription factor responsible for controlling the expression of Ecadherin and itself in a double feedback loop (Burk et al., 2008). Upregulation of mir-200c likely downregulates ZEB1 expression, induces E-cadherin expression, and reduces the motility of cells thus obviating EMT. NSCLC cell lines resistant to erlotinib display EMT while sensitive cells do not.

The coupling of the GEPR and microRNA predictor of response to EGFR inhibition is contemplated to increase the accuracy of prediction of solid tumor patients that will respond to EGFR inhibition in a clinical setting. This could yield significant benefit to the treatment of tumors particularly in those that display EGFR-dependent phenotypes but do not display known biomarkers of EGFR inhibitor response, such as pancreatic cancer (Tzeng et al., 2007).

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Certain genes (including gene products such as messenger RNAs (mRNAs)), are identified herein with reference to publicly-available database identifiers or numbers, as will be recognized by those skilled in the art. Information and sequences included in such databases are incorporated by reference as are equivalent and related sequences present in such databases, and all annotations present in such databases. Unless otherwise indicated or apparent the references to publicly-available databases are references to the most recent version of the database as of the filing date of this Application.

Some of the microRNAs (miRs) referenced herein are identified by miR IDs as used in the Sanger Institute miRBase Sequence Database (Sanger Database). Unless otherwise indicated or apparent, the references to the Sanger Database miR IDs are references to the miR IDs of the most recent version of the Sanger Database as of the filing date of this Application. The sequences cross-referenced in the Sanger Database are expressly incorporated by reference as are equivalent and related sequences present in Sanger Database or other public databases. Also expressly incorporated herein by reference are all annotations present in the Sanger Database associated with the miRNAs disclosed herein.

While the following terms are believed to be well understood by one of ordinary skill in the art, the following definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Throughout this document, various references are mentioned. All such references are incorporated herein by reference, including the references set forth in the following list.

### REFERENCES

1. Personeni N, Fieuws S, Piessevaux H, De Hertogh G, De Schutter J, Biesmans B, De Roock W, Capoen A, Debiec-Rychter M, Van Laethem J L, Peeters M, Humblet Y, Van Cutsem E, Tejpar S., “Clinical usefulness of EGFR gene copy number as a predictive marker in colorectal cancer patients treated with cetuximab: a fluorescent in situ hybridization study.” Clin Cancer Res. 2008 Sep. 15; 14(18):5869-76.
2. Razis E, Briasoulis E, Vrettou E, Skarlos D V, Papamichael D, Kostopoulos I, Samantas E, Xanthakis I, Bobos M, Galanidi E, Bai M, Gikonti I, Koukouma A, Kafiri G, Papakostas P, Kalogeras K T, Kosmidis P, Fountzilas G., “Potential value of PTEN in predicting cetuximab response in colorectal cancer: an exploratory study.” BMC Cancer. 2008 Aug. 13; 8:234.
3. Zhu C Q, da Cunha Santos G, Ding K, Sakurada A, Cutz J C, Liu N, Zhang T, Marrano P, Whitehead M, Squire J A, Kamel-Reid S, Seymour L, Shepherd F A, Tsao M S; National Cancer institute of Canada Clinical Trials Group Study BR.21. “Role of KRAS and EGFR as biomarkers of response to erlotinib in National Cancer Institute of Canada Clinical Trials Group Study BR.21.” J Clin Oncol. 2008 Sep. 10; 26(26):4268-75. Epub 2008 Jul. 14.
4. Hirsch F R, Herbst R S, Olsen C, Chansky K, Crowley J, Kelly K, Franklin W A, Bunn P A Jr, Varella-Garcia M, Gandara D R. “Increased EGFR gene copy number detected by fluorescent in situ hybridization predicts outcome in non-small-cell lung cancer patients treated with cetuximab and chemotherapy.” J Clin Oncol. 2008 Jul. 10; 26(20):3351-7.
5. De Roock W, Piessevaux H, De Schutter J, Janssens M, De Hertogh G, Personeni N, Biesmans B, Van Laethem J L, Peeters M, Humblet Y, Van Cutsem E, Tejpar S. “KRAS wild-type state predicts survival and is associated to early radiological response in metastatic colorectal cancer treated with cetuximab.”
6. Di Fiore F, Blanchard F, Charbonnier F, Le Pessot F, Lamy A, Galais M P, Bastit L, Killian A, Sesboue R, Tuech J J, Queuniet A M, Paillot B, Sabourin J C, Michot F, Michel P, Frebourg T. “Clinical relevance of KRAS mutation detection in metastatic colorectal cancer treated by Cetuximab plus chemotherapy.” Br J Cancer. 2007 Apr. 23; 96(8):1166-9. Epub 2007 Mar. 20.
7. Balko J M, Potti A, Saunders C, Stromberg A, Haura E B, Black E P. “Gene expression patterns that predict sensitivity to epidermal growth factor receptor tyrosine kinase inhibitors in lung cancer cell lines and human lung tumors.” BMC Genomics 2006; 7:289.
8. Balko, J M and Black, E P. “A gene expression predictor of response to EGFR-targeted therapy stratifies progression-free survival to cetuximab in KRAS wild-type metastatic colorectal cancer.” BMC Cancer 2009, 9:145.
9. Karapetis et al., The New England Journal of Medicine. 2008. 359:17 p 1757-1765.
10. Khambata-Ford S, Garrett C R, Meropol N J, et al. “Expression of epiregulin and amphiregulin and KRAS mutation status predict disease control in metastatic colorectal cancer patients treated with cetuximab.” J Clin Oncol 2007 25:3230-7.
11. Therasse P, Arbuck S G, Eisenhauer E A, Wanders J, Kaplan R S, Rubinstein L, Verweij J, Van Glabbeke M, van Oosterom A T, Christian M C, Gwyther S G. “New guidelines to evaluate the response to treatment in solid tumors.” European Organization for Research and Treatment of Cancer, National Cancer Institute of the United States, National Cancer Institute of Canada. J Natl Cancer Inst. 2000 Feb. 2; 92(3):205-16.
12. Cheng Y, Prusoff W H. “Relationship between the inhibition constant (K1) and the concentration of inhibitor which causes 50 percent inhibition (I50) of an enzymatic reaction”. Biochem Pharmacol (December 1973) 22 (23): 3099-108.
13. Parker & Barnes, Methods in Molecular Biology 106:247-283, 1999.
14. Hod, Biotechniques 13:852-854 (1992).
15. Weis et al., Trends in Genetics 8:263-264 (1992).
16. Schena et al., Proc. Natl. Acad. Sci. USA 93(2):106-149 (1996).
17. Ausubel et al., Current Protocols of Molecular Biology, John Wiley and Sons, 1997.
18. Rupp and Locker, “Methods for RNA extraction from paraffin embedded tissues are disclosed,” for example, in, Lab Invest. 56:A67 (1987).
19. De Andres et al., BioTechniques 18:42044 (1995).
20. Held et al., Genome Research 6:986-994 (1996).
21. T. E. Godfrey et al. J. Molec. Diagnostics 2: 84-91 (2000).
22. K. Specht et al., Am. J. Pathol. 158: 419-29 (2001).
23. Ding and Cantor, Proc. Natl. Acad. Sci. USA 100:3059-3064 (2003).
24. Liang and Pardee, Science 257:967-971 (1992).
25. Kawamoto et al., Genome Res. 12:1305-1312 (1999).
26. Oliphant et al., “Discovery of Markers for Disease” (Supplement to Biotechniques), June 2002.
27. Ferguson et al., Analytical Chemistry 72:5618 (2000).
28. Yang et al., Genome Res. 11:1888-1898 (2001).
29. Fukumura et al., Nucl. Acids. Res. 31(16) e94 (2003).
30. Velculescu et al., Science 270:484-487 (1995).
31. Velculescu et al., Cell 88:243-51 (1997).
32. Burk U, Schubert J, Wellner U, Schmalhofer O, Vincan E, Spaderna S et al (2008). “A reciprocal repression between ZEB1 and members of the miR-200 family promotes EMT and invasion in cancer cells.” EMBO Rep 9: 582-9.
33. Coldren C D, Helfrich B A, Witta S E, Sugita M, Lapadat R, Zeng C et al (2006). “Baseline gene expression predicts sensitivity to gefitinib in non-small cell lung cancer cell lines.” Mol Cancer Res 4: 521-8.
34. Filipowicz W, Bhattacharyya S N, Sonenberg N (2008). “Mechanisms of posttranscriptional regulation by microRNAs: are the answers in sight?” Nat Rev Genet 9:102-14.
35. Gendreau S B, Ventura R, Keast P, Laird A D, Yakes F M, Zhang W et al (2007). “Inhibition of the T790M gatekeeper mutant of the epidermal growth factor receptor by EXEL-7647.” Clin Cancer Res 13: 3713-23.
36. Lu J, Getz G, Miska E A, Alvarez-Saavedra E, Lamb J, Peck D et al (2005). “MicroRNA expression profiles classify human cancers.” Nature 435: 834-8.
37. Pao W, Miller V A (2005). “Epidermal growth factor receptor mutations, small-molecule kinase inhibitors, and non-small-cell lung cancer: current knowledge and future directions.” J Clin Oncol 23: 2556-68.
38. Park S M, Gaur A B, Lengyel E, Peter M E (2008). “The miR-200 family determines the epithelial phenotype of cancer cells by targeting the E-cadherin repressors ZEB1 and ZEB2.” Genes Dev 22: 894-907.
39. Reich M, Liefeld T, Gould J, Lerner J, Tamayo P, Mesirov JP (2006). “GenePattern 2.0.” Nat Genet 38: 500-1.
40. Simon R (2008). “The use of genomics in clinical trial design.” Clin Cancer Res 14: 5984-93.
41. Thomson S, Buck E, Petti F, Griffin G, Brown E, Ramnarine N et al (2005). “Epithelial to mesenchymal transition is a determinant of sensitivity of non-small-cell lung carcinoma cell lines and xenografts to epidermal growth factor receptor inhibition.” Cancer Res 65: 9455-62.
42. Tzeng C W, Frolov A, Frolova N, Jhala N C, Howard J H, Buchsbaum D J et al (2007). “Epidermal growth factor receptor (EGFR) is highly conserved in pancreatic cancer.” Surgery 141: 464-9.
43. Wiemer E A (2007). “The role of microRNAs in cancer: no small matter.” Eur J Cancer 43: 1529-44.
44. Yauch R L, Januario T, Eberhard D A, Cavet G, Zhu W, Fu L et al (2005). “Epithelial versus mesenchymal phenotype determines in vitro sensitivity and predicts clinical activity of erlotinib in lung cancer patients.” Clin Cancer Res 11: 8686-98.
45. Tusher V G, Tibshirani R, and Chu G. (2001). “Significance analysis of microarrays applied to the ionizing radiation response.” PNAS 98: 5116-5121.
46. The Gene Ontology Consortium. “Creating the gene ontology resource: design and implementation,” Genome Res 2001; 11(8):1425-33.

1-50. (canceled)

51. A method for predicting clinical outcome for a subject with a carcinoma comprising the steps of:
determining an RNA expression profile in the carcinoma of the subject; and
applying an algorithm for predicting a clinical outcome indicator from an RNA expression profile of a carcinoma to the RNA expression profile of the subject to predict a clinical outcome indicator of the subject,
wherein the RNA expression profile comprises at least ten predictive markers.

52. The method of claim 51, further comprising the steps of:
applying a second algorithm for predicting a second clinical outcome indicator from the RNA expression profile of a carcinoma to the RNA expression profile of the subject to predict a second clinical outcome indicator of the subject;
wherein the first clinical outcome indicator is EGFR inhibitor sensitivity or survival and wherein the second clinical indicator is EGFR inhibitor sensitivity or survival and wherein the first and second clinical outcome indicators are not the same clinical outcome indicator.

53. The method of claim 51, wherein the predictive markers are messenger RNA (mRNA) molecules.

54. The method of claim 51, wherein the predictive markers are microRNA (miR) molecules.

55. The method of claim 51, wherein the predictive markers comprise ten or more predictive markers from Table 1.

56. The method of claim 51, wherein the predictive markers comprise ten or more predictive markers from Table 5.

57. The method of claim 51, wherein the predictive markers comprise ten or more predictive markers from Table 1 and Table 5.

58. The method of claim 51, wherein the predictive markers comprise: mir-140, mir-628, mir-135b, mir-200b, mir-141, mir-200c, mir-205, mir-197, mir-224, mir-34c, mir-301a, mir-636, and mir-518f.

59. The method of claim 58, wherein the carcinoma is a non-small cell lung carcinoma; and wherein the clinical outcome indicator is EGFR inhibitor sensitivity.

60. The method of claim 59, wherein the EGFR inhibitor is erlotinib.

61. The method of claim 51, wherein the predictive markers comprise ten or more mRNAs selected from the group consisting of: DDR1, PTGES3, GNB2L1, DPYSL2, RAP1B, LGALS3BP, CD59, SNX17, IPO7, ARF4, GNAI3, NR4A1, SRF, MAPRE2, MAPK14, PRKCD, ARL2, ARL3, PIK3R3, RALBP1, RYK, YES1, IMPA1, SMAD2, CTNNBIP1, RASSF2, MAP2K4, NSMAF, ARRB2, TRIM38, HMOX1, TMED1, LAMA3, MAP3K5, PLCB4, ARHGAP29, GNB5, GNG11, VDR, RGS19, TRAF5, PIK3CA, CAMK1, PIK3C2B, PDE4D, ADCY9, ELMO1, RAB40B, DKK1, NR4A2, ITGAE, RARB, NCF4, GPR109B, GPSM2, BCL10, GNA15, VEGFA, NUDT11, GLRX2, GPR87, LOC644617, STAP2, RAB35, JAG2, PRKD2, FKBP8, CC2D1A, ARHGDIB, CSNK2B, ADRBK1, DPYSL3, SQSTM1, IGFBP4, GJA1, SNX1, ARAF, PRDX4, INPP4B, MST1R, NPAS2, ADORA1, SMURF2, ASB9, MAP2K6, CDS1, TULP3, PRKD1, ADORA2B, RAB3B, IL27RA, IL15, BRAF, PRKCH, STAT4, GRB14, 2RY2, GABRA5, INSL4, LPAR2, TNFSF9, PTK7, EMR1, PDE1C, IL15RA, CREM, TNFRSF1A, DGKD, ECOP, RAC1, RAB8A, TXN, MDK, RGL2, IRS2, TNFRSF10B, CHUK, PRKCI, RHOD, RND1, MAPK13, FYN, KLRG1, VEGFA, EGFR, EXT2, LANCL1, IGBP1, NEDD9, MMS19, AMFR, TTRAP, TACSTD2, BCR, RASA1, PDE10A, PDE4B, RAB36, BAG1, MAPK11, WNK1, GPR56, RHOB, RBM9, RHOQ, NUDT4, ATP2C1, GNAS, PDCD11, PKN2, CAMK2G, HMHAl, TIAM1, SRC, STX2, CAP1, DIXDC1, APLP2, ITGA6, IDE, RAB31, SNX5, TFG, DYNC1LI1, ARL5A, RAB25, PRDM4, RAB22A, P2RY5, TNFRSF21, GMIP, RAB17, EDN1, HS1BP3, OPN3, DAPP1, GPRC5C, GTPBP1, DLL3, and DEF8.

62. The method of claim 59, wherein the predictive markers comprise the following mRNAs: DDR1, PTGES3, GNB2L1, DPYSL2, RAP1B, LGALS3BP, CD59, SNX17, IPO7, ARF4, GNAI3, NR4A1, SRF, MAPRE2, MAPK14, PRKCD, ARL2, ARL3, PIK3R3, RALBP1, RYK, YES1, IMPA1, SMAD2, CTNNBIP1, RASSF2, MAP2K4, NSMAF, ARRB2, TRIM38, HMOX1, TMED1, LAMA3, MAP3K5, PLCB4, ARHGAP29, GNB5, GNG11, VDR, RGS19, TRAF5, PIK3CA, CAMK1, PIK3C2B, PDE4D, ADCY9, ELMO1, RAB40B, DKK1, NR4A2, ITGAE, RARB, NCF4, GPR109B, GPSM2, BCL10, GNA15, VEGFA, NUDT11, GLRX2, GPR87, LOC644617, STAP2, RAB35, JAG2, PRKD2, FKBP8, CC2D1A, ARHGDIB, CSNK2B, ADRBK1, DPYSL3, SQSTM1, IGFBP4, GJA1, SNX1, ARAF, PRDX4, INPP4B, MST1R, NPAS2, ADORA1, SMURF2, ASB9, MAP2K6, CDS1, TULP3, PRKD1, ADORA2B, RAB3B, IL27RA, IL15, BRAF, PRKCH, STAT4, GRB14, 2RY2, GABRA5, INSL4, LPAR2, TNFSF9, PTK7, EMR1, PDE1C, 1L15RA, CREM, TNFRSF1A, DGKD, ECOP, RAC1, RAB8A, TXN, MDK, RGL2, IRS2, TNFRSF10B, CHUK, PRKCI, RHOD, RND1, MAPK13, FYN, KLRG1, VEGFA, EGFR, EXT2, LANCL1, IGBP1, NEDD9, MMS19, AMFR, TTRAP, TACSTD2, BCR, RASA1, PDE10A, PDE4B, RAB36, BAG1, MAPK11, WNK1, GPR56, RHOB, RBM9, RHOQ, NUDT4, ATP2C1, GNAS, PDCD11, PKN2, CAMK2G, HMHA1, TIAM1, SRC, STX2, CAP1, DIXDC1, APLP2, ITGA6, IDE, RAB31, SNX5, TFG, DYNC1LI1, ARL5A, RAB25, PRDM4, RAB22A, P2RY5, TNFRSF21, GMIP, RAB17, EDN1, HS1BP3, OPN3, DAPP1, GPRC5C, GTPBP1, DLL3, and DEF8; wherein the carcinoma is a non-small cell lung carcinoma; and wherein the clinical outcome indicator is EGFR inhibitor sensitivity.

63. The method of claim 62, wherein the EGFR inhibitor is erlotinib.

64. The method of claim 62, wherein the predictive markers further comprise the following miRNAs: mir-140, mir-628, mir-135b, mir-200b, mir-141, mir-200c, mir-205, mir-197, mir-224, mir-34c, mir-301a, mir-636, and mir-518f

65. The method of claim 58, wherein the carcinoma is a pancreatic cancer; and wherein the clinical outcome indicator is EGFR inhibitor sensitivity.

66. The method of claim 65, wherein the EGFR inhibitor is cetuximab.

67. The method of claim 51, wherein the predictive markers comprise ten or more mRNAs selected from the group consisting of: DDR1, PRDX4, RYK, HMOX1, GNB5, PIK3CA, ELMO1, TNFRSF10B, GPR109B, PTK7, TNFRSF1A, RAC1, ECOP, RGL2, PRKC1, ATP2C1, GNAS, CAMK2G, ITGA6, P2RY5, PRKD2, CC2D1A, MAPK13, VEGFA, RHOB, and NUDT4.

68. The method of claim 65, wherein the predictive markers comprise the following mRNAs: DDR1, PRDX4, RYK, HMOX1, GNB5, PIK3CA, ELMO1, TNFRSF10B, GPR109B, PTK7, TNFRSF1A, RAC1, ECOP, RGL2, PRKC1, ATP2C1, GNAS, CAMK2G, ITGA6, P2RY5, PRKD2, CC2D1A, MAPK13, VEGFA, RHOB, and NUDT4; wherein the carcinoma is a pancreatic cancer; and wherein the clinical outcome indicator is EGFR inhibitor sensitivity.

69. The method of claim 68, wherein the EGFR inhibitor is cetuximab.

70. The method of claim 68, wherein the predictive markers further comprise the following miRNAs: mir-140, mir-628, mir-135b, mir-200b, mir-141, mir-200c, mir-205, mir-197, mir-224, mir-34c, mir-301a, mir-636, and mir-518f

71. A device for predicting a clinical outcome indicator for a carcinoma comprising probes for predictive markers for determining an RNA expression profile in a carcinoma of a subject, wherein the probes include probes for at least ten predictive markers selected from Table 1 and Table 5.
